(19) United States
(12) Patent Application Publication
(10) Pub. No.: US 2022/0098160 A1
(43) Pub. Date: Mar. 31, 2022

(54) NOVEL CDK 8/19 INHIBITORS

(71) Applicant: BIOCAD, St. Petersburg (RU)

(72) Inventors: Aleksei Leonidovich MINDICH, St. Petersburg (RU); Svetlana Leonidovna GORBUNOVA, St. Petersburg (RU); Aleksandra Vladimirovna POPKOVA, Leningradskaya Obl. (RU); Artsiom Evgenievich SHEKHAUTSOU, Byhov (BY); Andrei Ivanovich ALAFINOV, Leningradskaja Obl. (RU); Mariia Andreevna KASATKINA, Krasnodar (RU); Natalia Vladimirovna KOZHEMYAKINA, St. Petersburg (RU); Anna Sergeevna KUSHAKOVA, Leningradskaya Obl. (RU); Elena Aleksandrovna MAKSIMENKO, St. Petersburg (RU); Leonid Evgen`evich MIKHAYLOV, St. Petersburg (RU); Mariia Sergeevna MISHINA, St. Petersburg (RU); Anna Jur'evna CHESTNOVA, Leningradskaja Obl. G. (RU); Dmitry Valentinovich MOROZOV, Moscow (RU)

(21) Appl. No.: 17/052,078

(22) PCT Filed: Feb. 28, 2019
(86) PCT No.: PCT/RU2019/050021

§ 371 Date: Dec. 13, 2021

(30) Foreign Application Priority Data
Mar. 1, 2018 (RU) 2018107581

(51) Int. Cl. C07D 239/80; A61P 35/00; C07D 239/95; C07D 237/28; C07D 215/48; C07D 471/04

(52) U.S. Cl. CPC C07D 239/80; A61P 35/00; C07D 471/04; C07D 237/28; C07D 215/48; C07D 239/95

(57) ABSTRACT

The present invention relates to novel compounds of formula (1), which have the properties of a CDK8/19 inhibitor, and to a pharmaceutical composition containing said compounds, and to the use of said compounds and composition as pharmaceutical preparations for treating diseases or disorders.

### FIELD OF THE INVENTION

This invention relates to new inhibitors of cyclin-dependent protein kinases CDK8/19, methods for preparation thereof, pharmaceutical compositions containing such compounds, and to the use of such compounds or such compositions for the treatment of diseases or disorders.

### STATE OF THE ART

Cyclin-dependent protein kinase CDK8, along with CDK19 isoform, which is closely related thereto based on its structural and functional characteristics, is an oncogenic kinase that regulates transcription (Xu, W. & Ji, J. Y. (2011) Dysregulation of CDK8 and Cyclin C in tumorigenesis, J. Genet. Genomics 38, 439-452; Galbraith, M D, et al. (2010) CDK8: a positive regulator of transcription, Transcription. 1, 4-12; Firestein, R. & Hahn, Wsee prev.C. (2009) Revving the Throttle on an oncogene: CDK8 takes the driver seat, Cancer Res 69, 7899-7901). In contrast to the more well-known members of the CDK family (such as CDK1, CDK2 and CDK4/6), CDK8 does not play a role in the regulation of the cell cycle, however, the knockout of the CDK8 gene in embryonic stem cells leads to a halt in embryo development (Adler, A. S., et al. (2012) CDK8 maintains tumor de-differentiation and embryonic stem cell pluripotency, Cancer Res. 72, 2129-2139) because of its important role in the formation of the pluripotent stem cell phenotype (Firestein, R., et al. (2008) CDK8 is a colorectal cancer oncogene that regulates beta-catenin activity, Nature 455, 547-551). It should be noted that blocking CDK8 does not inhibit normal cell growth (Adler, A. S., et al. (2012) CDK8 maintains tumor differentiation and embryonic stem cell pluripotency, Cancer Res. 72, 2129-2139, Kapoor, A., et al. (2010) The histone variant macroH2A suppresses melanoma progression through regulation of CDK8, Nature 468, 1105-1109). The role of CDK8 in carcinogenesis is associated with its unique function as a regulator of several transcription factors (Xu, W. & Ji, J. Y. (2011) Dysregulation of CDK8 and Cyclin C in tumorigenesis, J. Genet. Genomics 38, 439-452). High expression of CDK8 was detected during colon cancer (Firestein, R., et al. (2010) CDK8 expression in 470 colorectal cancers in relation to beta-catenin activation, other molecular alterations and patient survival, Int. J. Cancer 126, 2863-2873) and melanoma (Kapoor, A., et al. (2010) The histone variant macroH2A suppresses melanoma progression through regulation of CDK8, Nature 468, 1105-1109), although, with these types of cancer, increased expression of CDK8 is observed in ˜50% of cases. A similar situation is observed with breast cancer (E. Broude, et al. (2015) Expression of CDK8 and CDK8-interacting genes as potential biomarkers in breast cancer, Curr. Cancer Drug Targets, 15 (8), 739-749). Increased CDK8 expression is associated with poor prognosis for colon cancer (Gyorffy, B., et al. (2010) An online survival analysis tool to rapidly assess the effect of 22,277 genes on breast cancer prognosis using microarray data of 1, 809 patients, Breast Cancer Res. Treat. 123, 725-731).

Known mechanisms associated with CDK8 during cancer include upregulation of the Wnt/[beta] catenin pathway (Kapoor, A., et al. (2010) The histone variant macroH2A suppresses melanoma progression through regulation of CDK8, Nature 468, 1105-1109; Alarcon, C., et al. (2009) Nuclear CDKs drive Smad transcriptional activation and turnover in BMP and TGF-beta pathways, Cell 139, 757-769), growth factor-induced transcription (DiDonato, J. A., et al. (2012) NF-kappaB and the link between inflammation and cancer, Immunol. Rev. 246, 379-400), and the TGF-beta signaling pathway (Acharyya, S., et al. (2012) A CXCL1 paracrine network links cancer chemoresistance and metastasis, Cell 150, 165-178). It has also been shown that CDK8 can support the pluripotent phenotype of embryonic stem cells, which can be associated with the cancer stem cell phenotype (Firestein, R., et al. (2008) CDK8 is a colorectal cancer oncogene that regulates beta-catenin activity, Nature 455, 547-551). Chemotherapeutic drugs that cause DNA damage induce TNF-α, an activator of the NF-kB transcription factor (Fabian et al. (2005) A small molecule-kinase interaction map for clinical kinase inhibitors, Nat. Biotechnol. 23, 329-336), in endothelial cells and in other stromal elements of the tumor microenvironment. Stromal TNF-α activator affects tumor cells, where it induces NF-kB-mediated production of CXCL1 and CXCL2 cytokines, which contribute to the survival and growth of tumor cells. CXCL1/2 attract myeloid cells to the tumor by binding to the CXCR2 receptor on the surface of myeloid cells. Myeloid cells then secrete small calcium-binding proteins S 100A8 and A9, which are associated with the processes of chronic inflammation and tumor growth. S 100A8/9 affect tumor cells, promoting metastasis thereof as well as survival secondary to chemotherapy (Huang, et al. (2012) MED 12 Controls the response to multiple cancer drugs through regulation of TGF-b receptor signaling, Cell 151, 937-950).

Currently, it is important to search for new compounds inhibiting cyclin-dependent protein kinases CDK8/19.

### DESCRIPTION OF THE INVENTION

The following are the definitions of terms used in the description of this invention.

“Alkyl” means an aliphatic hydrocarbon linear or branched group with the chain containing 1 to 12 carbon atoms or, more preferably, 1 to 6 carbon atoms. “Branched” means that the alkyl chain has one or more “lower alkyl” substituents. Examples of alkyl groups include, but are not limited to, methyl, ethyl, n-propyl, iso-propyl, n-butyl, iso-butyl, sec-butyl, tert-butyl, n-pentyl, 2-pentyl, 3-pentyl, neo-pentyl, and n-hexyl. Alkyl may have substituents, which may be the same or different.

“Aryl” means an aromatic monocyclic or polycyclic system comprising 6 to 14 carbon atoms or, preferably, 6 to 10 carbon atoms. Examples of aryl groups include, but are not limited to, phenyl, naphthyl, anthranyl, etc. Aryl may have cyclic substituents, which may be the same or different. Aryl can be annelated with a non-aromatic ring system or heterocycle.

“Alkyloxy” or “Alkoxy” means an alkyl-O-group, in which alkyl is defined in this section. Examples of alkoxy groups include, but are not limited to, methoxy, ethoxy, n-propoxy, iso-propoxy, and n-butoxy.

“Amino group” means R′R″N-group, substituted or unsubstituted with optionally the same substituents R′ and R″.

“Alkylsulfonyl” (—S(O)₂—C₁-C₆ alkyl) means “alkyl” as defined above, attached to the corresponding moiety of the molecule via a sulfonyl group —SO₂—. Examples of alkylsulfonyls include, but are not limited to, methylsulfonyl, ethylsulfonyl, etc.

“Lower alkyl” means a linear or branched alkyl with 1 to 4 carbon atoms.

“Halo” or “Halogen” (Hal) means fluorine, chlorine, bromine or iodine.

“Heterocycle,” “heterocyclyl,” or “heterocyclic ring” means a monocyclic or polycyclic system containing from 3 to 11 carbon atoms, in which one or more carbon atoms are substituted for a heteroatom, such as nitrogen, oxygen, or sulfur. The heterocycle may be fused with aryl or heteroaryl. The heterocycle may have one or more substituents, which may be the same or different. The nitrogen and sulfur atoms in the heterocycle can be oxidized to N-oxide, S-oxide, or S-dioxide. The heterocycle may be saturated, partially unsaturated, or unsaturated. Examples of heterocycles include, but are not limited to, azetidine, pyrrolidine, piperidine, 2,8-diazaspiro[4.5]decane, piperazine, morpholine, etc.

“Heteroaryl” means an aromatic monocyclic or polycyclic system comprising 5 to 11 carbon atoms or, preferably, from 5 to 10 carbon atoms, in which one or more carbon atoms are substituted for a heteroatom, such as nitrogen, sulfur, or oxygen. The nitrogen atom in the heteroaryl group can be oxidized to N-oxide. Heteroaryl may have one or more substituents, which may be the same or different. Heteroaryl representatives include pyrrolyl, furanyl, thienyl, pyridyl, pyrazinyl, pyrimidinyl, pyridazinyl, isoxazolyl, isothiazolyl, tetrazolyl, oxazolyl, thiazolyl, pyrazolyl, furazanyl, triazolyl, 1,2,4-thiadiazolyl, quinoxalinyl, phthalazinyl, imidazo[1,2-a]pyridinyl, imidazo[2,1-b]thiazolyl, benzofurazanil, indolyl, azainindolyl, benzimidazolyl, benzothiazenyl, quinolinyl, imidazolyl, pyrazolyl, thienopyridyl, quinazolinyl, naphthyridinyl, thienopyrimidine, pirollopyridinyl, imidazopyridyl, isoquinolinyl, benzoazaindolyl, 1,2,4-triazinyl, thienopyrrolyl, furopyrrolyl, etc.

“Partially unsaturated” means a ring system comprising at least one double or triple bond. The term “partially unsaturated” refers to rings having a plurality of saturation sites, but does not include aryl and heteroaryl systems, as defined above.

The term “oxo,” as used herein, refers to an ═O radical.

“Substituent” means a chemical radical that binds to a molecular skeleton (scaffold, moiety).

“Solvate” means a molecular complex of a compound according to this invention, including pharmaceutically acceptable salts thereof, with one or more solvent molecules. Such solvent molecules are those which are commonly used in pharmaceuticals and are known to be harmless to the recipient, for example water, ethanol, ethylene glycol, and the like. Other solvents can be used as intermediate solvates during preparation of more desirable solvates, such as methanol, methyl-tert-butyl ether, ethyl acetate, methyl acetate, (S)-propylene glycol, (R)-propylene glycol, 1,4-butanediol, and the like.

The term “hydrate” refers to a complex in which the solvent molecule is water.

Solvates and/or hydrates preferably exist in the crystalline form.

The terms “bond,” “chemical bond,” or “single bond” refer to a chemical bond between two atoms or two groupings (groups, moieties), if two atoms connected by such bond are considered part of a larger substructure.

The term “protective group” refers to groups used to block the reactivity of such functional groups as amino groups, carboxyl groups or hydroxyl groups. Examples of protective groups include, but are not limited to, tert-butoxycarbonyl (BOC), benzyloxycarbonyl (CBZ), 2-(trimethylsilyl)ethoxy methyl acetal (SEM), trialkylsilyl, alkyl(diaryl)silyl, or alkyl.

The term “excipient” is used herein to describe any ingredient other than the compound(s) according to this invention.

“Pharmaceutical composition” means a composition comprising a compound of the invention and at least one excipient. The excipient may be selected from the group consisting of pharmaceutically acceptable and pharmacologically compatible fillers, solvents, diluents, carriers; auxiliary, distributing, and perceptive means; delivery vehicles, such as preservatives, stabilizers, fillers, grinders, moisturizers, emulsifiers, suspending agents, thickeners, sweeteners, perfumes, fragrances, antibacterial agents, fungicides, lubricants, prolonged delivery regulators, the choice and ratio of which depends on the nature, method of administration, and dosage. Examples of the suspending agents include ethoxylated isostearyl alcohol, polyoxyethylene, sorbitol and sorbitol ether, microcrystalline cellulose, aluminum metahydroxide, bentonite, agar-agar, and tragacanth, as well as mixtures of these substances. Protection against microorganisms can be achieved using a variety of antibacterial and antifungal agents, such as parabens, chlorobutanol, sorbic acid, etc. The composition may also include isotonic agents, such as sugars, sodium chloride, etc. The prolonged action of the composition can be achieved by using agents that slow down the absorption of the active principle, such as aluminum monostearate and gelatin. Examples of suitable carriers, solvents, diluents, and delivery vehicles are water, ethanol, polyalcohols, and also mixtures thereof; vegetable oils (such as olive oil) and injectable organic esters (such as ethyl oleate). Examples of excipients are lactose, milk sugar, sodium citrate, calcium carbonate, calcium phosphate, etc. Examples of grinders and dispensers are starch, alginic acid and its salts, silicates, etc. Examples of lubricants are magnesium stearate, sodium lauryl sulfate, talc, and high molecular weight polyethylene glycol. The pharmaceutical composition for oral, sublingual, transdermal, intramuscular, intravenous, subcutaneous, local, or rectal administration of the active principle (alone or in combination with another active principle) can be administered to animals and humans in a standard administration form as a mixture with traditional pharmaceutical carriers. Suitable standard administration forms include oral (e.g., tablets, gelatin capsules, pills, powders, granules, chewing gums, and oral solutions or suspensions), sublingual, and buccal administration forms; aerosols, implants, local, transdermal, subcutaneous, intramuscular, intravenous, intranasal, or intraocular administration forms; and rectal administration forms.

“Pharmaceutically acceptable salt” means relatively non-toxic salts of the compounds according to this invention. Salts of the compounds provided herein may be prepared from inorganic or organic acids and bases. Examples of salts thus obtained are hydrochlorides, hydrobromides, sulfates, bisulfates, phosphates, nitrates, acetates, oxalates, valeriates, oleates, palmitates, stearates, laurates, borates, benzoates, lactates, tosylates, citrates, maleates, fumarates, succinates, tartrates mesylates, malonates, salicylates, propionates, ethanesulfonates, benzenesulfonates, sulfamates and the like; salts of sodium, potassium, ammonium, calcium, magnesium, iron, zinc, copper, manganese and aluminum, salts of primary, secondary and tertiary amines, substituted amines, including naturally substituted amines, cyclic amines, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, ethylenediamine, glucosamine, methylglucamine, theobromine, purines, piperazine, piperidine, piperidine, piperidine, and N-ethyl piperidine (for a detailed description of the properties of such salts, see: Berge S. M., et al., “Pharmaceutical Salts,” J. Pharm Sci 1977, 66: 1-19).

“Medicinal product (preparation)” is a substance (or a mixture of substances in the form of a pharmaceutical composition) in the form of tablets, capsules, injections, ointments and other formulations intended to restore, correct or alter physiological functions in humans and animals, as well as treatment and prevention of diseases, diagnostics, anesthesia, contraception, cosmetology, etc.

“To treat,” “treatment,” and “therapy” refer to a method of alleviating or eliminating a biological disorder, and/or at least one of its related symptoms. The term “to alleviate” a disease, illness, or condition means to decrease the severity and/or frequency of occurrence of the symptoms of such disease, disorder, or condition. In addition, references to “treatment” contained herein include references to therapeutic, palliative, and preventive therapy.

In one aspect, the subject of treatment (or patient) is a mammal and, preferably, a human subject. Such subject can be male or female, and be of any age.

The term “disorder” means any condition that can be improved as a result of treatment according to this invention. The definition of this term includes chronic and acute disorders or diseases, involving pathological conditions causing predisposition of a mammal to the occurrence of such disorder. Non-limiting examples of the treatable diseases include oncological diseases, such as breast cancer, triple negative breast cancer (TNBC), ovarian cancer, metastatic ovarian cancer, gastric cancer, metastatic gastric cancer, endometrial cancer, salivary gland, lung, kidney, colon, and colorectal cancer, melanoma, metastatic melanoma, cancer of the thyroid gland, pancreas, prostate or bladder; blood cancers, leukemia, acute myeloid leukemia, and lymphoid malignancies; neural, glial, astrocytal, hypothalamic and other granular, macrophage, epithelial, stromal and blastocellular disorders; inflammatory, angiogenic and immunological disorders.

A “therapeutically effective amount” is the amount of a therapeutic agent administered during treatment that will relieve to some extent one or more of the symptoms of the disease being treated.

Unless provided otherwise, words “to have,” “to include,” and “to comprise” or variations thereof, such as “has,” “having,” “includes,” “including,” “comprises,” or “comprising,” as used in the present description and subsequent claims, should be construed as an inclusion of the specified items or group of items, and not as an exclusion of any other item or group of items.

### DETAILED DESCRIPTION OF THE INVENTION

In one embodiment, the present invention relates to a compound of formula I.

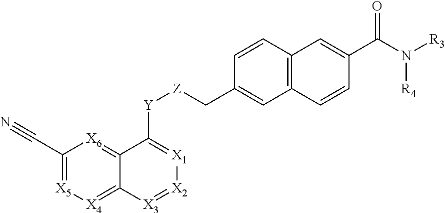

where X₁ represents CR₁, N;
X₂ represents CR₂, N;
X₃, X₄, X₅, X₆ each independently represent CH, N;
R₁ represents H, Hal, C₁-C₆ alkyl, C₃-C₇ cycloalkyl;
R₂ represents H, Hal, C₁-C₆ alkyl, —OR₁₀, —NR₁₁R₁₂,
R₃ and R₄ each independently represent H; C₁-C₆ alkyl unsubstituted or substituted with one or more halogens; C₁-C₆ alkoxy C₁-C₆ alkyl; C₃-C₇ cycloalkyl; C₃-C₇ cycloalkyl C₁-C₃ alkyl; C₆-C₁₀ aryl unsubstituted or substituted with one or more substituents selected from Hal, C₁-C₆ alkyl unsubstituted or substituted with one or more halogens, C₁-C₆, alkoxy; 5-10 membered heteroaryl with 1-4 heteroatoms selected from N, O, and/or S unsubstituted or substituted with one or more substituents selected from Hal, C₁-C₆ alkyl unsubstituted or substituted with one or more halogens, C₁-C₆ alkoxy; 5-6 membered heterocyclyl with 1-2 heteroatoms selected from N, O, and/or S unsubstituted or substituted with one or more substituents selected from Hal; —OH; C₁-C₆ alkoxy; C₁-C₆ alkyl unsubstituted or substituted with one or more halogens, C₁-C₆ alkoxy; or
R₃ and R₄, together with the nitrogen atom to which they are attached, may represent a 4-7 membered heterocyclyl with 1-2 heteroatoms selected from N and/or O, which may be unsubstituted or substituted with one or more substituents selected from the oxo group; —OH; C₁-C₆ alkoxy; C₁-C₆ alkyl unsubstituted or substituted with one or more halogens, C₁-C₆ alkoxy;
Y represents —N(R₁₃)—, —O—, —S—, or —C(O)—;
Z represents —C(R₁₄)₂— or —C(O)—;
R₁₀ represents H, C₁-C₆ alkyl, C₁-C₆ acyl;
R₁₁, R₁₂ each independently represent H, C₁-C₆ alkyl, C₃-C₇ cycloalkyl;
R₁₃ represents H, C₁-C₆ alkyl, C₁-C₆ acyl;
R₁₄ each independently represents H, C₁-C₆ alkyl, C₃-C₇ cycloalkyl; not including compounds where X₁, X₃ represent N; X₂, X₄, X₅, X₆ represent CH; R₃ and R₄ together with the nitrogen atom to which they are attached represent

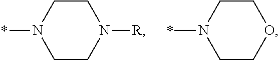

where R represents H, C₁-C₆ alkyl.

In yet another embodiment, the present invention relates to a compound of formula I, wherein

if X₁ is CR₁, X₂ is CR₂, and X₃ is N, then X₄, X₅, X₆ represent CH;
if X₁ is CR₁, and X₂, X₃ are N, then X₄, X₅, X₆ represent CH;
if X₂ is CR₂, and X₁, X₃ are N, then X₄, X₅, X₆ represent CH;
if X₁ is CR₁, and X₂, X₄ are N, then X₃, X₅, X₆ represent CH;
if X₁ is CR₁, X₂ is CR₂, and X₃, X₅ are N, then X₄, X₆ represent CH;
if X₁ is CR₁, X₂ is CR₂, and X₃, X₄ are N, then X₅, X₆ represent CH;
if X₁ is CR₁, X₂ is CR₂, and X₃, X₆ are N, then X₄, X₅ represent CH.

In yet another embodiment, the present invention relates to a compound of formula I, where Y is —NH—, —O—, or —S—; and Z is —CH₂—.

In yet another embodiment, the present invention relates to a compound of formula II:

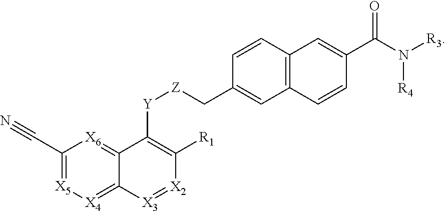

where X₂ represents CR₂, N;
X₃, X₄, X₅, X₆ each independently represent CH, N;
R₁ represents H, Hal, C₁-C₆ alkyl, C₃-C₇ cycloalkyl;
R₂ represents H, Hal, C₁-C₆ alkyl, —OR₁₀, —NR₁₁R₁₂,
R₃ and R₄ each independently represent H; C₁-C₆ alkyl unsubstituted or substituted with one or more halogens; C₁-C₆ alkoxy C₁-C₆ alkyl; C₃-C₇ cycloalkyl; C₃-C₇ cycloalkyl C₁-C₃ alkyl; C₃-C₁₀ aryl unsubstituted or substituted with one or more substituents selected from Hal, C₁-C₆ alkyl unsubstituted or substituted with one or more halogens, C₁-C₆ alkoxy; 5-10 membered heteroaryl with 1-4 heteroatoms selected from N, O, and/or S unsubstituted or substituted with one or more substituents selected from Hal, C₁-C₆ alkyl unsubstituted or substituted with one or more halogens, C₁-C₆ alkoxy; 5-6 membered heterocyclyl with 1-2 heteroatoms selected from N, O, and/or S unsubstituted or substituted with one or more substituents selected from Hal; —OH; C₁-C₆ alkoxy; C₁-C₆ alkyl unsubstituted or substituted with one or more halogens, C₁-C₆ alkoxy; or
R₃ and R₄ together with the nitrogen atom to which they are attached may represent a 4-7 membered heterocyclyl with 1-2 heteroatoms selected from N and/or O, which may be unsubstituted or substituted with one or more substituents selected from the oxo group; —OH; C₁-C₆ alkoxy; C₁-C₆ alkyl unsubstituted or substituted with one or more halogens, C₁-C₆ alkoxy;
Y represents —N(R₁₃)—, —O—, —S—, or —C(O)—;
Z represents —C(R₁₄)₂— or —C(O)—;
R₁₀ represents H, C₁-C₆ alkyl, C₁-C₆ acyl;
R₁₁, R₁₂ each independently represent H, C₁-C₆ alkyl, C₃-C₇ cycloalkyl;
R₁₃ is H, C₁-C₆ alkyl, C₁-C₆ acyl;
R₁₄ each independently represents H, C₁-C₆ alkyl, C₃-C₇ cycloalkyl.

In yet another embodiment, the present invention relates to a compound of formula II, where Y represents —NH—, —O—, or —S—; and Z represents —CH₂—.

In yet another embodiment, the present invention relates to a compound of formula I or II, where R₃ and R₄ each independently represent H; C₁-C₆ alkyl unsubstituted or substituted with one or more halogens; C₁-C₆ alkoxy C₁-C₆ alkyl; C₃-C₇ cycloalkyl, C₃-C₇ cycloalkyl C₁-C₃ alkyl; C₆-C₁₀ aryl representing phenyl unsubstituted or substituted with one or more substituents selected from Hal, C₁-C₆ alkyl unsubstituted or substituted with one or more halogens, C₁-C₆ alkoxy; 5-10 membered heteroaryl with 1-4 heteroatoms selected from N, O, and/or S representing pyridine or pyrimidine unsubstituted or substituted with one or more substituents selected from Hal, C₁-C₆ alkyl unsubstituted or substituted with one or more halogens, C₁-C₆ alkoxy; 5-6 membered heterocyclyl with 1-2 heteroatoms selected from N, O, and/or S, which represents 4-morpholinyl, 1-piperazinyl, 1-pyrrolidinyl, 1-piperidinyl unsubstituted or substituted with one or more substituents selected from Hal; —OH; C₁-C₆ alkoxy; C₁-C₆, alkyl unsubstituted or substituted with one or more halogens, C₁-C₆, alkoxy; or

R₃ and R₄ together with the nitrogen atom to which they are attached may represent a 4-7 membered heterocyclyl with 1-2 heteroatoms selected from N and/or O, which represents morpholinyl, piperazinyl, pyrrolidinyl, piperidinyl, azetidine, which can be unsubstituted or substituted with one or more substituents selected from oxo group; —OH; C₁-C₆ alkoxy; C₁-C₆ alkyl unsubstituted or substituted with one or more halogens, C₁-C₆, alkoxy.

In yet another embodiment, the present invention relates to a compound of formula III:

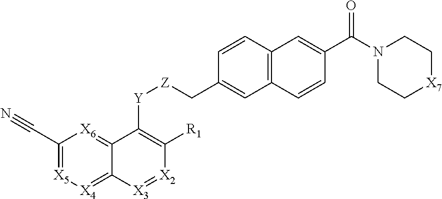

where X₂ represents CR₂, N;
X₃, X₄, X₅, X₆ each independently represent CH, N;
R₁ represents H, Hal, C₁-C₆ alkyl, C₃-C₇ cycloalkyl;
R₂ represents H, Hal, C₁-C₆ alkyl, —OR₁₀, —NR₁₁R₁₂,
X₇ represents —N(R₁₅), —O—;
Y represents —N(R₁₃)—, —O—, —S—, or —C(O)—;
Z represents —C(R₁₄)₂— or —C(O)—;
R₁₀ represents H, C₁-C₆ alkyl, C₁-C₆ acyl;
R₁₁, R₁₂ each independently represent H, C₁-C₆ alkyl, C₃-C₇ cycloalkyl;
R₁₅ represents H, C₁-C₆ alkyl unsubstituted or substituted with one or more halogens, C₁-C₆ alkoxy.

In yet another embodiment, the present invention relates to a compound of formula III, where Y represents —NH—, —O—, or —S—; and Z represents —CH₂—.

In yet another embodiment, the present invention relates to a compound of formula II or III, where if X₂ is CR₂ and X₃ is N, then X₄, X₅, X₆ represent CH;

if X₂, X₃ are N, then X₄, X₅, X₆ represent CH;
if X₂, X₄ are N, then X₃, X₅, X₆ represent CH;
if X₂ is CR₂, and X₃, X₅ are N, then X₄, X₆ represent CH;
if X₂ is CR₂, and X₃, X₄ are N, then X₅, X₆ represent CH;
if X₂ is CR₂, and X₃, X₆ are N, then X₄, X₅ represent CH.

In yet another embodiment, the present invention relates to a compound of formula I.1:

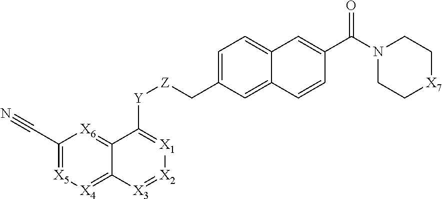

where X₁ represents CR₁, N;
X₂ represents CR₂, N;
X₃, X₄, X₅, X₆ each independently represent CH, N;
R₁ represents H, Hal, C₁-C₆ alkyl, C₃-C₇ cycloalkyl;
R₂ represents H, Hal, C₁-C₆ alkyl, —OR₁₀, —NR₁₁R₁₂,
X₇ represents —N(R₁₅), —O—;
Y represents —N(R₁₃)—, —O—, —S—, or —C(O)—;
Z represents —C(R₁₄)₂— or —C(O)—;
R₁₀ represents H, C₁-C₆ alkyl, C₁-C₆ acyl;
R₁₁, R₁₂ each independently represent H, C₁-C₆ alkyl, C₃-C₇ cycloalkyl;
R₁₅ represents H, C₁-C₆ alkyl unsubstituted or substituted with one or more halogens; not including compounds where X₁, X₃ represent N; X₂, X₄, X₅, X₆ represent CH; R₃ and R₄ together with the nitrogen atom to which they are attached represent

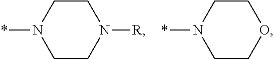

where R represents H, C₁-C₆ alkyl.

In yet another embodiment, the present invention relates to a compound of formula I.2:

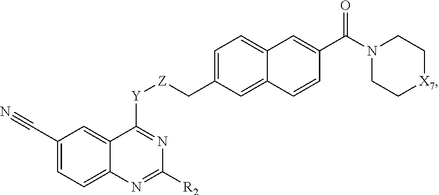

where X₇, Y, Z, R₂ have the above values;
not including compounds, where X₁, X₃ are N; X₂, X₄, X₅, X₆ are CH; R₃ and R₄ together with the nitrogen atom to which they are attached represent

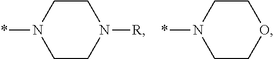

where R represents H, C₁-C₆ alkyl.

In yet another embodiment, the present invention relates to a compound of formula II.1:

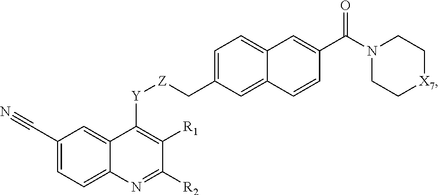

where X₇, Y, Z, R₁, R₂ have the above values.

In yet another embodiment, the present invention relates to a compound of formula II.2:

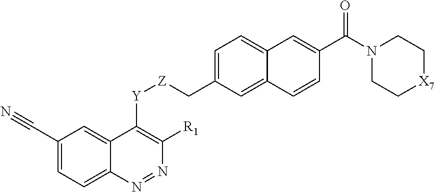

where X₇, Y, Z, R₁ have the above values.

In yet another embodiment, the present invention relates to a compound of formula II.3:

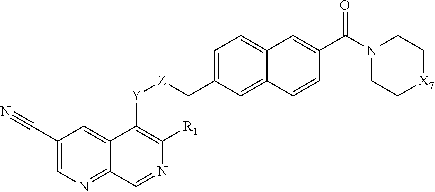

where X₇, Y, Z, R₁ have the above values.

In yet another embodiment, the present invention relates to a compound of formula II.4:

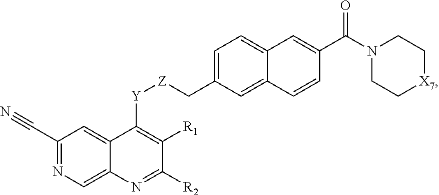

where X₇, Y, Z, R₁, R₂ have the above values.

In yet another embodiment, the present invention relates to a compound of formula II.5:

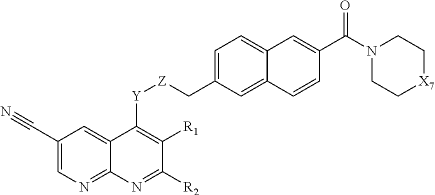

where X₇, Y, Z, R₁, R₂ have the above values.

In yet another embodiment, the present invention relates to a compound of formula II.6:

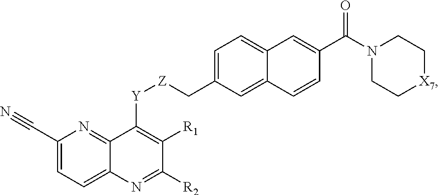

where X₇, Y, Z, R₁, R₂ have the above values.

The compounds described in the present invention can be obtained and/or used in the form of pharmaceutically acceptable salts. The types of pharmaceutically acceptable salts include, but are not limited to, acid salts formed by reacting the free base compound with a pharmaceutically acceptable inorganic acid, such as hydrochloric, hydrobromic, sulfuric, nitric, phosphoric, metaphosphoric acids, etc.; or with an organic acid, such as acetic, propionic, caproic, cyclopentane propionic, glycolic, pyruvic, lactic, malonic, succinic, malic, maleic, fumaric, trifluoroacetic, tartaric, citric, benzoic, 3-(4-hydroxybenzoic)benzoic, cinnamic, mandelic acids, methane-sulfonic acid, ethane-sulfonic acid, 1,2-ethane-disulfonic acid, 2-hydroxyethane-disulfonic acid, benzene-sulfonic acid, toluene-sulfonic acid, 2-naphthalene-sulfonic acid, 4-methylbicyclo[2.2.2]oct-2-en-1-carboxylic, glucoheptonic, 4,4′-methylene-bis-3-hydroxy-2-en-1-carboxylic, 3-phenylpropionic, trimethylacetic, tert-butylacetic, laurylsulfuric, gluconic, glutamic, hydroxy-naphthoic, salicylic, stearic, muconic acids, etc.

The corresponding counter-ions of pharmaceutically acceptable salts can be studied and identified using various methods, including but not limited to ion exchange chromatography, ion chromatography, capillary electrophoresis, induction plasma binding, atomic absorption spectroscopy, mass spectrometry, or any combination thereof.

The salts are reduced using at least one of the following methods: filtration, precipitation with a precipitant with subsequent filtration, evaporation of the solvent or, in the case of aqueous solutions, lyophilization. It should be understood that mentioning a pharmaceutically acceptable salt includes acid-addition salts with solvent or crystalline forms of such salts, in particular solvates or polymorphs. Solvates contain a stoichiometric or non-stoichiometric amount of solvent and can be formed during the crystallization process with pharmaceutically acceptable solvents such as water, ethanol, and the like. Hydrates are formed when the solvent is water, and alcoholates are formed when the solvent is alcohol. Solvates of the compounds described in this patent can be easily prepared or formed in the methods described in the present invention. In addition, the compounds provided for by the present invention may exist in unsolvated as well as solvated forms. In general, solvated forms are considered equivalent to unsolvated forms when describing the compounds and methods provided by the present invention.

The compounds described in the present invention can be presented in various forms including but not limited to those listed above: structureless forms, ground forms, and nanoparticles. In addition, the compounds described in the present invention include crystalline forms, also known as polymorphs. Polymorphs include crystals with different structures of the same elemental composition of the compound. Typically, polymorphs demonstrate a different X-ray diffraction pattern, different infrared spectra, melting points, different densities, hardness, crystalline form, optical and electrical properties, stability and solubility. Various factors, such as type of recrystallization solvent, degree of crystallization, and storage temperature may determine the dominance of a single crystalline form.

Screening and characterization of pharmaceutically acceptable salts, polymorphs and/or solvates can be carried out by a number of methods including but not limited to thermal analysis, X-ray diffraction, spectroscopy, vapor sorption, and microscopy. Thermal analysis methods are aimed at studying thermochemical decomposition or thermophysical processes including but not limited to polymorphic transitions, and such methods are used to analyze the relationship between polymorphic forms to determine the mass loss, to find the glass transition temperature, or to study compatibility with the filler. Such methods include, without limitation, differential scanning calorimetry (DSC), modulating differential scanning calorimetry (MDSK), thermogravimetric analysis (TGA), thermogravimetric and infrared analysis (TG/IR). Crystallographic methods include but are not limited to those listed above: single crystal and powder diffractometers and synchrotron sources. Various utilized spectroscopic methods include but are not limited to determining the Raman spectrum (Raman scattering), FTIR, UVIS, and NMR (liquid and solid). Various microscopy methods include but are not limited to the following: polarized light microscopy, scanning electron microscopy (SEM) with X-ray energy dispersion analysis (EDX), electron microscopy scanning in a natural environment with EDX (in a gas or water vapor atmosphere), JR microscopy, and Raman microscopy.

In yet another embodiment, the present invention relates to compounds selected from the group which includes:

| Number | Formula | Name |
|---|---|---|
| 1 | 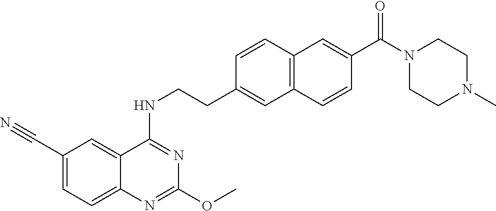 [SMILES: COc1nc(NCCc2ccc3cc(C(=O)N4CCN(C)CC4)ccc3c2)c2cc(C#N)ccc2n1] | 2-dimethoxy-4-((2-(6-(4- methylpiperazine-l-carbonyl) naphth-2-yl)ethyl)amino) quinazoline-6-carbonitryl |
| 2 | 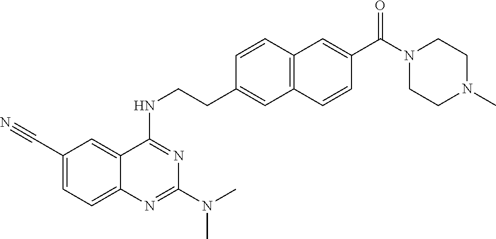 [SMILES: CN1CCN(C(=O)c2ccc3cc(CCNc4nc(N(C)C)nc5ccc(C#N)cc45)ccc3c2)CC1] | 2-(dimethylamino)-4-((2-(6- (4-methylpiperazine-1- carbonyl) naphth-2- yl)ethyl)amino) quinazoline- 6-carbonitryl |
| 3 | 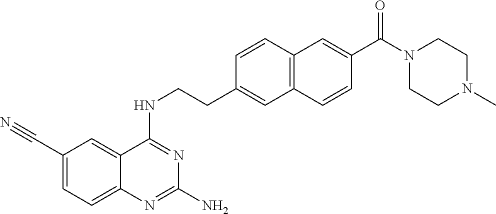 [SMILES: CN1CCN(C(=O)c2ccc3cc(CCNc4nc(N)nc5ccc(C#N)cc45)ccc3c2)CC1] | 2-amino-4-((2-(6-(4- methylpiperazine-l-carbonyl) naphth-2-yl)ethyl)amino) quinazoline-6-carbonitryl |
| 4 | 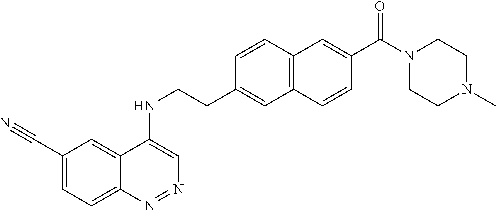 [SMILES: CN1CCN(C(=O)c2ccc3cc(CCNc4cnnc5ccc(C#N)cc45)ccc3c2)CC1] | 4-((2-(6-(4-methylpiperazine- 1-carbonyl)naphth-2- yl)ethyl)amino) cinnoline-6- carbonitryl |
| 5 | 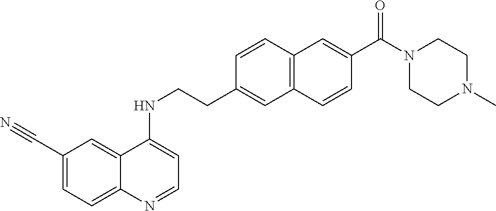 [SMILES: CN1CCN(C(=O)c2ccc3cc(CCNc4ccnc5ccc(C#N)cc45)ccc3c2)CC1] | 4-((2-(6-(4-methylpiperazine- 1-carbonyl)naphth-2- yl)ethyl)amino) quinoline-6- carbonitryl |
| 5a | 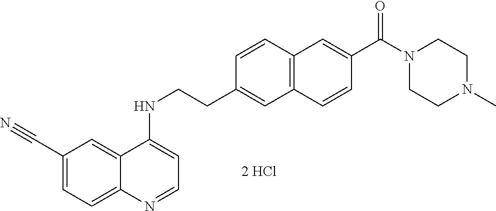 [SMILES: CN1CCN(C(=O)c2ccc3cc(CCNc4ccnc5ccc(C#N)cc45)ccc3c2)CC1] | 4-((2-(6-(4-methylpiperazine- 1-carbonyl)naphth-2- yl)ethyl)amino) quinoline-6- carbonitryl dihydrochloride |
| 6 | 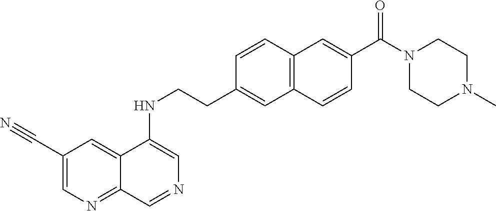 [SMILES: CN1CCN(C(=O)c2ccc3cc(CCNc4cncc5ncc(C#N)cc45)ccc3c2)CC1] | 5-((2-(6-(4-methylpiperazine- 1-carbonyl)naphth-2- yl)ethyl)amino) 1,7- naphthyridine-3-carbonitryl |
| 7 | 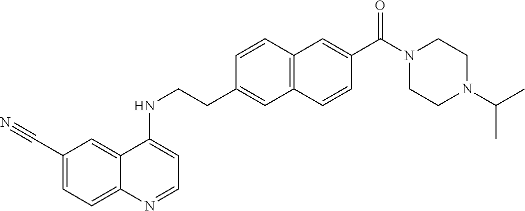 [SMILES: CC(C)N1CCN(C(=O)c2ccc3cc(CCNc4ccnc5ccc(C#N)cc45)ccc3c2)CC1] | 4-((2-(6-isopropylpiperazine- 4-carbonyl)naphth-2- yl)ethyl)amino) quinoline-6- carbonitryl |
| 8 | 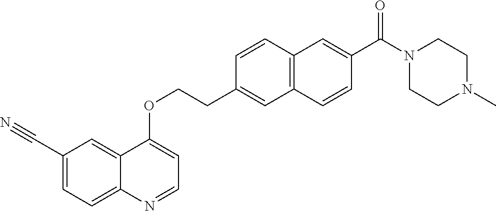 [SMILES: CN1CCN(C(=O)c2ccc3cc(CCOc4ccnc5ccc(C#N)cc45)ccc3c2)CC1] | 4-(2-(6-(4-methylpiperazine- 1-carbonyl)naphth-2- yl)ethoxy) quinoline-6- carbonitryl |
| 9 | 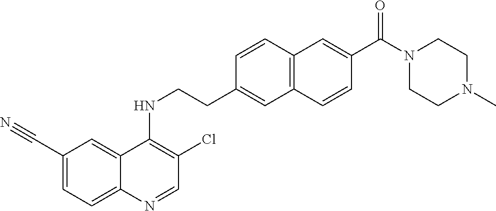 [SMILES: CN1CCN(C(=O)c2ccc3cc(CCNc4c(Cl)cnc5ccc(C#N)cc45)ccc3c2)CC1] | 3-chloro-4-((2-(6-(4- methylpiperazine-1- carbonyl)naphth-2- yl)ethyl)amino) quinoline-6- carbonitryl |
| 9a | 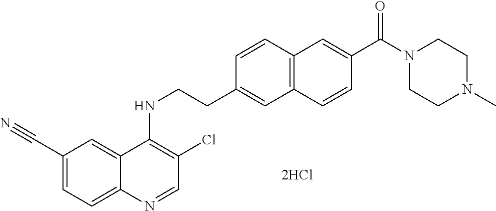 [SMILES: CN1CCN(C(=O)c2ccc3cc(CCNc4c(Cl)cnc5ccc(C#N)cc45)ccc3c2)CC1] | 3-chloro-4-((2-(6-(4- methylpiperazine-1- carbonyl)naphth-2- yl)ethyl)amino) quinoline-6- carbonitryl dihydrochloride |
| 11 | 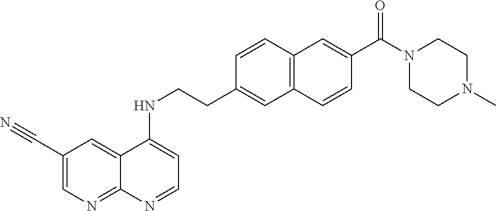 [SMILES: CN1CCN(C(=O)c2ccc3cc(CCNc4ccnc5ncc(C#N)cc45)ccc3c2)CC1] | 5-((2-(6-(4-methylpiperazine- 1-carbonyl)naphth-2- yl)ethyl)amino)-1,8- naphthyridin-3-carbonitryl |
| 12 | 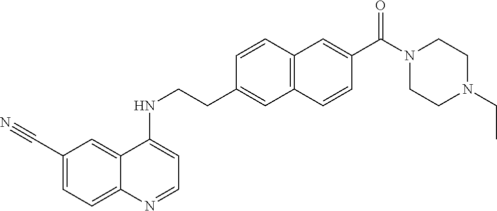 [SMILES: CCN1CCN(C(=O)c2ccc3cc(CCNc4ccnc5ccc(C#N)cc45)ccc3c2)CC1] | 4-((2-(6-ethylpiperazine-4- carbonyl)naphth-2-yl)ethyl) amino)quinoline-6-carbonitryl |
| 13 | 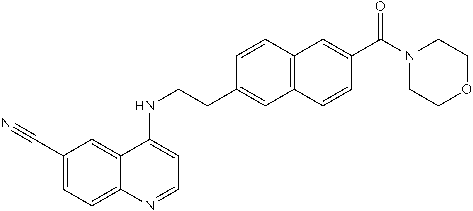 [SMILES: N#Cc1ccc2nccc(NCCc3ccc4cc(C(=O)N5CCOCC5)ccc4c3)c2c1] | 4-((2-(6-morpholine-4- carbonyl)naphth-2-yl)ethyl) amino)quinoline-6-carbonitryl |
| 17 | 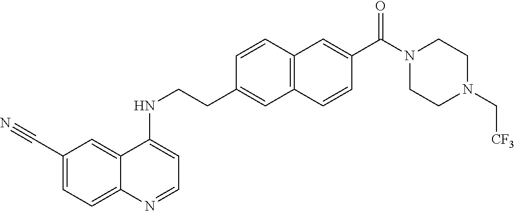 [SMILES: N#Cc1ccc2nccc(NCCc3ccc4cc(C(=O)N5CCN(CC(F)(F)F)CC5)ccc4c3)c2c1] | 4-((2-(6-(4-(2,2,2- trifluoroethyl)piperazine-4- carbonyl)naphth-2-yl)ethyl) amino)quinoline-6-carbonitryl |
| 18 | 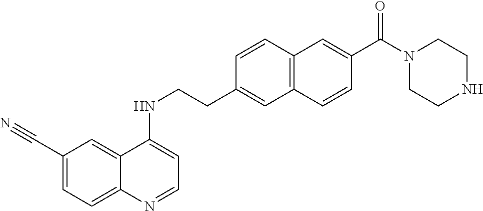 [SMILES: N#Cc1ccc2nccc(NCCc3ccc4cc(C(=O)N5CCNCC5)ccc4c3)c2c1] | 4-((2-(6-(piperazine-1- carbonyl)naphth-2-yl)ethyl) amino)quinoline-6-carbonitryl |
| 19 | 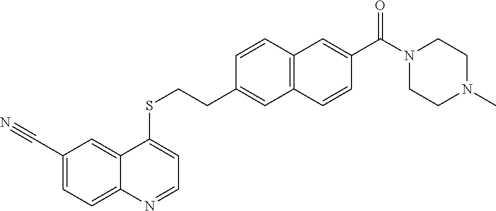 [SMILES: CN1CCN(C(=O)c2ccc3cc(CCSc4ccnc5ccc(C#N)cc45)ccc3c2)CC1] | 4-((2-(6-(4-methylpiperazine- 1-carbonyl)naphth-2- yl)ethyl)thio) quinoline-6- carbonitryl |

The present invention also relates to a method for inhibiting the biological activity of cyclin-dependent protein kinases (CDK8/19) in a subject by putting said cyclin-dependent protein kinases (CDK8/19) in contact with a compound according to this invention.

In one embodiment, the present invention relates to a pharmaceutical composition comprising a therapeutically effective amount of the compound described herein, or a pharmaceutically acceptable salt or solvate thereof, and one or more pharmaceutically acceptable excipients. In yet another embodiment of the invention, the pharmaceutical composition according to this invention is intended for the prevention or treatment of a disease or disorder mediated by activating cyclin-dependent protein kinases (CDK8/19). In yet another embodiment of the invention, the pharmaceutical composition of this invention is intended for the prevention or treatment of a disease or disorder mediated by activating cyclin-dependent protein kinases (CDK8/19), wherein the disease or disorder mediated by the activation of said cyclin-dependent protein kinases (CDK8/19) is an oncological disease or a blood cancer. In yet another embodiment, the pharmaceutical composition according to this invention is intended for the prevention or treatment of colorectal cancer, melanoma, metastatic melanoma, breast cancer, triple negative breast cancer (TNBC), prostate cancer, metastatic ovarian cancer, metastatic gastric cancer, leukemia, acute myeloid leukemia, and pancreatic cancer (PC).

The pharmaceutical composition according to the present invention may contain, for example, from about 10% to about 100% of the active ingredients, preferably, from about 20% to about 60% of the active ingredients. It is understood that the content of the active ingredient or ingredients in an individual dose of each dosage form does not necessarily constitute an effective amount, since the required effective amount can be achieved by introducing several standard dosage forms.

A typical composition is obtained by mixing a compound according to the present invention with one or more excipients. Examples of such excipients include, but are not limited to, diluents, carriers, and fillers. Suitable carriers, diluents, and fillers are well known to those skilled in this field and include substances, such as carbohydrates, waxes, water-soluble and/or swellable polymers, hydrophilic or hydrophobic substances, gelatin, oils, solvents, water and the like. A specific utilized carrier, diluent or filler will depend on the means and the purpose for which the compound of the present invention is used. Solvents are generally selected from the solvents recognized as safe for administration to a mammal by those skilled in the art. In general, safe solvents include aqueous solvents, such as water, and other solvents that are water-soluble or miscible with water. Suitable aqueous solvents include water as the main component, as well as ethanol, propylene glycol, polyethylene glycols (e.g., PEG400, PEG300), etc., and mixtures thereof. The compositions may also include one or more buffers, stabilizing agents, surfactants, wetting agents, lubricants, emulsifiers, suspending agents, preservatives, antioxidants, matting agents, glidants, processing aids, dyes, sweeteners, fragrances, flavors and other additives known to promote a good appearance of the drug (i.e., the compound according to the present invention or its pharmaceutical composition) or to help produce a pharmaceutical product (i.e., drug).

Pharmaceutical compositions may also include salts, solvates and hydrates of the compounds according to the present invention, or a stabilized form of the compound (e.g., a complex with a cyclodextrin derivative or other known complex formation agent).

The pharmaceutical compositions according to the present invention are generally suitable for oral administration. Oral administration of drugs means taking a medication by mouth (lat. per os, oris), or swallowing the medication. The compounds according to the present invention can also be administered buccally, lingually, or sublingually, so that the compound enters the bloodstream directly from the oral cavity.

Dosage forms suitable for oral, buccal, lingual, or sublingual administration include solid, semi-solid and liquid systems, such as tablets; granules; soft or hard capsules containing multi- or nanoparticles, liquids or powders; lozenges (including those filled with liquid); chewing forms; gels; rapidly soluble dosage forms; films; suppositories; sprays; as well as buccal/mucoadhesive patches.

Liquid pharmaceutical forms include suspensions, solutions, syrups, and elixirs. Such pharmaceutical dosage forms can be used as fillers in soft or hard capsules (e.g., made of gelatin or hydroxypropyl methyl cellulose) and usually contain a carrier, such as water, ethanol, polyethylene glycol, propylene glycol, methyl cellulose or a suitable oil and one or more emulsifiers and/or suspending agents. Liquid pharmaceutical dosage forms can also be made by reconstituting a solid compound, for example, from a sachet.

The compounds according to the present invention may also be administered parenterally. As used herein, the term “parenteral administration” of a pharmaceutical composition includes any route of administration that is characterized by a physical disruption of the integrity of the subject's tissue and administration of the pharmaceutical composition through such tissue disruption, which usually leads to a direct entry into the bloodstream, muscle, or internal organ. Thus, parenteral administration includes, but is not limited to, administration of a pharmaceutical composition by injection, through a surgical incision, via a non-surgical wound penetrating into the tissue, etc. Specifically, it is anticipated that parenteral administration includes, but is not limited to, subcutaneous, intraperitoneal, intramuscular, intravenous, intraarterial, intrathecal, intraventricular, intraurethral, intracranial, intraarticular injection or infusions, as well as renal dialysis infusion procedures. An intratumoral delivery, such as intratumoral injection, may also be useful. Regional perfusion is also provided.

The dosage forms of pharmaceutical compositions suitable for parenteral administration usually contain the active ingredient in combination with a pharmaceutically acceptable carrier, such as sterile water or sterile isotonic solution. Such dosage forms can be prepared and packaged in a form suitable for bolus administration or for continuous administration. Injectable dosage forms can be manufactured and packaged in a standard dosage form, such as ampoules or multi-dose containers containing a preservative. The dosage forms for parenteral administration include, but are not limited to, suspensions, solutions, emulsions in oil or aqueous bases, pastes, etc.

The pharmaceutical dosage forms can be used for immediate and/or modified release. Modified release dosage forms include delayed, extended, pulsed, controlled, targeted, and programmed release.

In one embodiment, the present invention relates to a method for treating a disease or disorder mediated by the activation of cyclin-dependent protein kinases (CDK8/19), which comprises administration of the therapeutically effective amount of a compound, pharmaceutically acceptable salt, or a pharmaceutical composition according to the present invention to a subject in need of such treatment.

In yet another embodiment, the present invention relates to a method for treating a disease or disorder mediated by the activation of cyclin-dependent protein kinases (CDK8/19), which is an oncological disease or blood cancer, which comprises administration of a therapeutically effective amount of the compound described herein, or a pharmaceutically acceptable salt, or a pharmaceutical composition thereof according to this invention to a subject in need of such treatment.

In yet another embodiment, the present invention relates to the treatment method described above, wherein the oncological disease or blood cancer is selected from the group consisting of colorectal cancer, melanoma, metastatic melanoma, breast cancer, triple negative breast cancer (TNBC), prostate cancer, metastatic ovarian cancer, metastatic stomach cancer, leukemia, acute myeloid leukemia, and pancreatic cancer (PC).

It is understood that the compounds according to this invention can be used as part of the treatment methods as described above, or can be used as part of treatment as described above, and/or can be used for manufacturing medications for treatment as described above.

The compounds that are inhibitors of CDK8/19 can be used as part of the treatment methods as described above, as a monotherapy, or in combination with surgery, radiation therapy, or medication therapy.

The terms “co-administration,” “co-administered,” “in combination with,” or “together with,” as used herein in reference to these compounds containing one or more therapeutic agents, are intended to mean, reference or include the following:
simultaneous administration of such combination of the compound according to this invention and a therapeutic agent to a patient in need of treatment, wherein such components are formulated together in one dosage form, from which they are released almost simultaneously,
simultaneous administration of such combination of the compound according to this invention and a therapeutic agent to a patient in need of treatment, wherein such components are formulated separately in the different dosage forms, however, the administration of such components occurs almost at the same time to a specific patient, after which they are released almost simultaneously,
sequential administration of such combination of the compound according to this invention and a therapeutic agent to a patient in need of treatment, wherein such components are formulated separately from each other in the separate dosage forms, which are received sequentially by a specific patient with a significant time interval between each administration, after which said components are released at different times; and
sequential administration of such combination of the compound according to this invention and a therapeutic agent to a patient in need of treatment, wherein such components are formulated together in a single dosage form, from which the release of these components occurs in a controlled manner, after which they are simultaneously, sequentially, or jointly released at the same and/or different time, while each part can be administered in one or different ways.

As known to those skilled in the art, the therapeutic effective dosages may vary when using drugs as part of the combined treatment. Methods for experimentally determining therapeutically effective dosages of the drugs and other agents for use as part of combined treatment regimens are described in the literature. For example, the use of uniform dosing (i.e., administration of more frequent and smaller doses to minimize toxic side effects) is described in the literature. In addition, combined treatment includes periodic treatment, which begins and stops at various times in accordance with the patient's treatment plan. In the combined therapy described in this patent, the dosages of the co-administered compounds will undoubtedly vary depending on the type of adjuvant used, specifics of the utilized drug, disease or condition being treated, etc.

In addition, the compounds described herein can also be used in combination with the procedures that can provide additive or synergistic benefits to the patient. By way of example only, patients are expected to receive therapeutic and/or prophylactic benefit by utilizing the methods described in this patent, wherein the pharmaceutical composition(s) of the compound described in this invention and/or combinations with other therapy methods are combined with a genetic study to determine whether an object is a carrier of a mutant gene, for which the correlation with certain diseases or conditions is known.

The above medication therapy may include the administration of one or more anti-cancer agents. Examples of anticancer agents include, without limitation, any of the following agents: alkylating agents, alkyl sulfonates, nitrosoureas or triazenes; antimetabolites; hormonal agents or hormone antagonists; platinum compounds; antitumor antibiotics; topoisomerase inhibitors.

Examples of antimetabolites include, but are not limited to, folic acid analogues (e.g., methotrexate, trimerexate, pemetrexed, pralatrexate, altitrexed, calcium levofolinate) or pyrimidine analogues (e.g., cytarabine, tegafur, fluorouracil, capecitabine, phloxuridine, azinocytin, azacycytin, sapacitabine, elacitarabine, doxyfluuridine), or purine analogues (e.g., mercaptopurine, thioguanine, pentostatin, fludarabine, cladribine, nonlarabin, azathioprine, clofarabin), or asparaginase.

Examples of alkylating agents include, without limitation, mehloroetamin, cyclophosphamide, chlorambucil, menfalan, bendamustine, geksametilimelamine, thiotepa, busulfan, carmustine, lomustine, laromustin, semustine, streptozocin, dacarbazine, ifosfamide, improsulfan, mitobronitol, mitolaktol, nimustine, ranimustine, temozolomide, threosulfan, carbochion, apazihion, fotemustine, altretamine, glufosfamide, pipobromane, trophosphamide, uramustine, euphosphamide, VAL-083.

Examples of hormonal agents and hormone antagonists include, but are not limited to, prednisone, prednisolone, hydroxyprogesterone caproate, megestrol acetate, medroxyprogesterone acetate, diethylstilbestrol, estradiol, tamoxifen, testosterone propionate, fluoxymesterone, flutamide, leuprolide, abarelix, abiraterone, bicalutamide, buserelin, calusterone, chlorotrianizen, degarelix, dexamethasone, fluocortolone, fulvestrant, goserelin, histrelin, leuprorelin, mitotan, nafarelin, nandrolone, nilutamide, octreotide, raloxifene, thyrotropin-alpha, toremifene, triptorelin, diethylstilbestrol, acolbifen, danazole, deslorelin, epithiostanol, orteronel, enzalutamide, aminoglutetimide, anastrozole, exemestane, fadrozole, letrozole, testolactone, and formestane.

Examples of platinum compounds include, but are not limited to, cisplatin, carboplatin, oxaliplatin, eptaplatin, myriplatin hydrate, lobaplatin, nedaplatin, picoplatin, and satraplatin.

Examples of antitumor antibiotics include, but are not limited to, doxorubicin, daunorubicin, idarubicin, carubicin, valrubicin, zorubicin, aclarubicin, pyrarubicin, nemorubicin, amrubicin, epirubicin, bleomycin, dactinomycin, plicamycin, peplomycin, mitomycin C, zinostatin, and streptozocin.

Examples of topoisomerase inhibitors include, but are not limited to, irinotecan, topotecan, belotecan, teniposide, etoposide, voreloxin, and amonafide.

Examples of anticancer agents include but are not limited to any of the following agents: microtubule-acting drugs, such as taxanes (e.g., paclitaxel, docetaxel, cabazitaxel, tesetaxel); vinca alkaloids (e.g., vinorelbine, vinblastine, vincristine, vindesine, vinflunine); mitogen-activated protein kinase signaling inhibitors (e.g., U0126, PD98059, PD184352, PD0325901, ARRY-142886, SB239063, SP600125, BAY 43-9006, Wortmannin or LY294002); mTOR inhibitors (e.g. sirolimus, temsirolimus, everolimus, ridaforolimus); antibodies (e.g., rituximab, trastuzumab, alemtuzumab, besilesomab, cetuximab, denosumab, ipilimumab, bevacizumab, pertuzumab, nivolumab, ofatumumab, panitumumab, tositumomab, katumaksomab, elotuzumab, epratuzumab, farletuzumab, mogamulizumab, netsitumumab, nimotuzumab, obinutuzumab, okaratuzumab, oregovomab, ramucirumab, rilotumumab, siltuximab, tocilizumab, zalutumumab, zanolumab, matuzumab, dalotuzumab, onartuzumab, racotumomab, tabalumab, abituzumab); kinase inhibitors (fosmatanib, entosplenib, erlotinib, imatinib, lapatinib, nilotinib, pazopanib, vemurafenib, gefitinib, krizotinib, dazatinib, regorafenib, ruxolitinib, sorafenib, sunitinib, vandetanib, bosutinib, axitinib, afatinib, alisertib, dabrafenib, dakomitinib, dinaciclib, dovitinib, nintedanib, lenvatinib, linifanib, linsitinib, masitinib, motesanib, neratinib, orantinib, pontatinib, radotinib, tipifarnib, tivantinib, tivozanib, trametinib, apatinib, ibrutinib, akalabrutinib, kobimetinib, fedratinib, brivanib alaninate, cediranib, cabozantinib, ikotinib, cipatinib, rigosertib, pimasertib, buparlisib, idelalisib, midostaurin, perifosin, and tesevatinib); photosensitizers (e.g., thalaporfin, temoporfin, sodium porphymer); cytokines (e.g., aldesleukin, interferon alpha, interferon alpha-2a, interferon alpha-2b, celmoleykin, tasonermin, recombinant interleukin-2, oprelvekin, and recombinant interferon beta-1a); vaccines (e.g., pitsibanil, sipuleucel-T, vitespen, emepepimut-S, oncoVAX, rindopepimut, troVAX, MGN-1601, and MGN-1703); bisanthrene, decitabine, mitoxantrone, procarbazine, trabectin, amsacrine, brostallicin, miltefosine, romidepsin, plitidepsin, eribulin, ixabepilone, fosbretabulin, denileukin diftitox, ibritumomab tiuxetan, prednimustine, trastuzumab emtansine, estramustine, gemtuzumab ozogamicin, aflibercept, oportuzumab monatox, cintredekin besudotox, edotreotide, inotuzumab ozogamicin, naptumab estafenatox, vintafolid, brentuximab vedotin, bortezomib, ixazomib, carfilzomib, lenalidomide, thalidomide, pomalidomide, zoledronic acid, ibandronic acid, pamidronic acid, alitretinoin, tretinoin, peretinoin, bexarotene, tamibaroten, imiquimod, lentinan, mifamurtid, romurtid, pegaspargaza, pentostatin, endostatin, sizofiran, vismodegib, vorinostat, entinostat, panobinostat, celecoxib, tsilengitid, etanidazole, ganetespib, idronoksil, iniparib, lonidamine, nimorazole, procodazole, taschinimod, telotristate, belinostat, thimalphazine, tirapazamine, tosedostat, trabedersen, ubenimex, valspodar, gendicin, reolizin, retaspimycin, trebananib, and virulizin.

In one embodiment, the present invention relates to the use of the compound described herein, or a pharmaceutically acceptable salt thereof, or a pharmaceutical composition according to this invention for treating a disease or disorder mediated by activating cyclin-dependent protein kinases (CDK8/19) in a subject in need of such treatment.

In yet another embodiment, the present invention relates to the use of a compound described herein, or a pharmaceutically acceptable salt thereof, or a pharmaceutical composition according to this invention in a subject in need of such treatment for the treatment of a disease or disorder mediated by activating cyclin-dependent protein kinases (CDK8/19), representing an oncological disease or blood cancer.

In yet another embodiment, the present invention relates to the use of a compound described above, or a pharmaceutically acceptable salt thereof, or a pharmaceutical composition according to this invention for the treatment of an oncological disease or blood cancer, which is selected from the group consisting of colorectal cancer, melanoma, metastatic melanoma, breast cancer, triple negative breast cancer (TNBC), prostate cancer, metastatic ovarian cancer, metastatic gastric cancer, leukemia, acute myeloid leukemia, and pancreatic cancer (PC), in a subject in need of such treatment. In any of the above treatments, the subject may be a human.

The compounds according to the present invention will be administered in an amount effective to treat the condition in question, i.e., in doses and for periods of time necessary to achieve the desired result. The therapeutically effective amount may vary depending on such factors as the specific condition being treated, patient's age, gender and weight, and whether the administration of these compounds is an independent treatment or is carried out in combination with one or more additional treatments.

Drug regimens can be adjusted to provide the optimum desired response. For example, a single dose may be administered, several divided doses may be administered over time, or a dose may be proportionally reduced or increased depending on the severity of the therapeutic situation. The most useful is the manufacture of a standard dosage form of the oral compositions to enable easy administration and uniform dosage. The standard dosage form, as used herein, refers to physically discrete units suitable as single doses for patients/subjects undergoing treatment. Each unit contains a predetermined amount of the active compound, calculated to produce the desired therapeutic effect in combination with the desired pharmaceutical carrier.

In addition, it is important to understand that for any particular patient, specific administration schemes should be adjusted after some time according to individual needs and the discretion of the healthcare professional in charge of administering or monitoring the administration of the compositions. Note that the concentration ranges provided in this description are only given as an example and are not intended to limit the scope or practice of the claimed compositions. In addition, the dosage regimen for the compositions according to this invention can be based on various factors, including the type of disease, age, weight, gender, health condition of the patient, severity of the condition, route of administration, and the particular utilized compound according to the present invention. Therefore, the dosage regimen can vary widely, but can be determined on a regular basis using standard methods. For example, doses may be adjusted based on pharmacokinetic and pharmacodynamic parameters, which may include clinical effects such as toxic effects or laboratory values. Thus, the present invention encompasses an individual dose increase, which is determined by a qualified specialist. Determination of the required dose and modes are well known in the relevant field of technology and will be clear to those skilled in the art after familiarization with the ideas disclosed herein.

As a rule, the doses used to treat an adult are usually in the range of 0.02-5,000 mg per day, or from about 1-1,500 mg per day.

When the patient's condition improves, a maintenance dose is administered, if necessary. Subsequently, the dosage or frequency of administration, or both, may be reduced, depending on the symptoms, to a level at which an improved condition of the disease, disorder, or condition is maintained. Patients may, however, require periodic treatment over time for any relapse of symptoms.

The above spectrum is only tentative, since the number of variables related to individual treatment regimen is extensive, and significant deviations from these recommended values are very common. These dosages can be changed depending on many variables, including but not limited to, the activity of the utilized compound, disease, or condition being treated, route of administration, as well as the needs of the individual subject, severity of the disease or condition being treated, and the opinion of the attending physician.

All publications, patents, and patent applications referred to in this specification are incorporated herein by reference. Although the aforementioned invention has been described in sufficient detail, those skilled in the art will understand based on the ideas disclosed in this invention that certain changes and modifications can be made without departing from the spirit and scope of the attached embodiments.

For a better understanding of the invention, the following examples are provided. These examples are for illustrative purposes only and should not be construed as limiting the scope of the invention in any form.

The compounds and processes of the present invention will be better understood in conjunction with the following synthesis schemes, which demonstrate the methods by which the compounds according to the present invention can be obtained. The starting products can be obtained from commercial sources or obtained using conventional methods from the prior art, known to those skilled in the art. It will also be apparent to those having ordinary skills in the art that the steps of selectively introducing and removing protection, as well as procedures for carrying out these steps, can be performed in different order, depending on the nature of the substituents, to successfully complete the synthesis presented below.

Abbreviations utilized herein, including those shown in illustrative schemes and the following examples, are well known to those skilled in the art. Some of the abbreviations used are as follows:

NMP—N-methyl pyrrolidone

### DMFA—N,N-dimethylformamide

DMSO—dimethyl sulfoxide
THE—tetrahydrofuran

### DMAA—N,N-dimethylacetamide

DIPEA—diisopropylethylamine
DIAD—diisopropyl azodicarboxylate
Pd(PPh₃)₄-tetrakis(triphenylphosphine)palladium(0)
BINAP—(±)-2,2′-bis(diphenylphosphino)-1,1′-dinaphthalene
CDI—carbonyldiimidazole
MTBE—methyl-tert-butyl ether

### NBS—N-Bromosuccinimide

### AIBN—Azobisisobutyronitrile

DMAP—4-dimethylaminopyridine
EDC×HCl—1-ethyl-3-(3-dimethylaminopropyl)carbodiimide hydrochloride.

Preparative purification of the synthesized compounds was performed using an AKTA Explorer 100 Air chromatograph with UV detection in the range from 254 to 360 nm. Separation was carried out in a gradient mode at different A and B eluent ratios.

| Method | Solvent A | Solvent B | Column |
|---|---|---|---|
| A | 1.8% aqueous | Acetonitrile | Inertsil_ODS3 30 mm × |
| | solution of | | 250 mm × 10 μm |
| | formic acid | | (GL Sciences) |
| B | 0.1% aqueous | Acetonitrile | Inertsil_ODS3 30 mm × |
| | solution of | | 250 mm × 10 μm |
| | formic acid | | (GL Sciences) |
| C | 0.1% aqueous | Acetonitrile | Pursuit XRs C18 30 |
| | solution of | | mm × 250 mm × 10 |
| | formic acid | | μm (Agilent) |
| D | 0.1% aqueous | 10% solution | Pursuit XRs C18 30 |
| | solution of | of acetonitrile | mm × 250 mm × |
| | formic acid | in methanol | 10 μm (Agilent) |

### EXAMPLES

### Example 1. Obtaining Intermediate Compounds 7.2, 12.2, 13.2, and 17.2

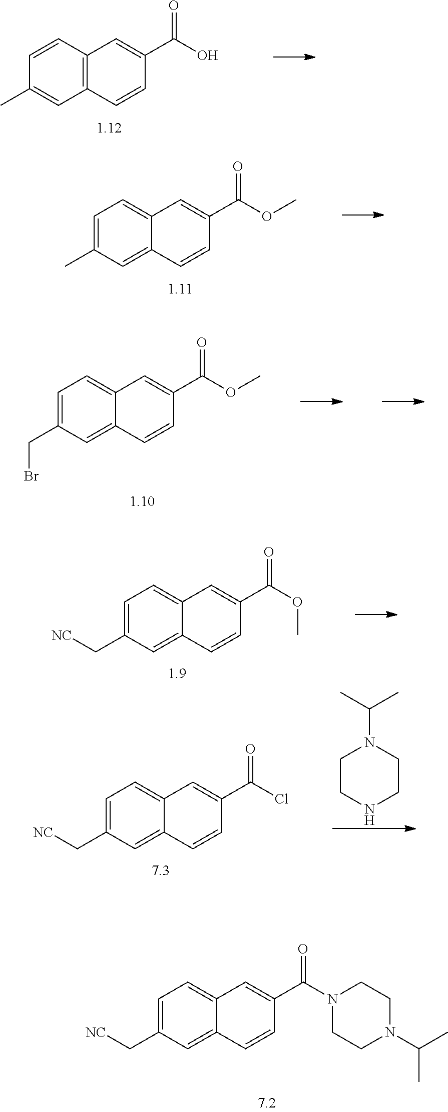

Step 1. SOCl₂ (1.64 g, 13.8 mmol) was added dropwise to 6-methylnaphthalene-2-carboxylic acid 1.12 (10.0 g, 53.7 mmol) suspended in 85 ml of methanol. After boiling for 1.5 h, SOCl₂ (660 mg, 5.5 mmol) was added dropwise. After stirring while boiling for 1 h, the solvent was removed under vacuum. Product 1.11 was obtained in the form of a light-yellow powder for use at the next step without any additional purification. Yield—10.3 g (98%).

Step 2. NBS (4.58 g, 25.7 mmol) and AIBN (370 mg, 2.3 mmol) were added to compound 1.11 (10.0 g, 49.9 mmol) suspended in 43 ml of carbon tetrachloride. After holding at 78° C. for 1.5 hours, the reaction mixture was cooled to 60° C. and more NBS (4.58 g, 25.7 mmol) and AIBN (370 mg, 2.3 mmol) were added. After holding at 63.5° C. for 1.5 h, the reaction mixture was cooled to 35° C. and 65 ml of hexane were added to it. The mixture was stirred for 2 hours at room temperature, the precipitate was filtered off, washed with hexane and a water-ethanol mixture (ratio—7:3), and dried in vacuum until the weight was no longer changing. Product 1.10 was obtained in the form of a yellow powder and used during the next step without additional purification. Yield—7.4 g (53%).

Step 3. KCN (2.07 g, 31.8 mmol) was added to a solution of compound 1.10 (7.4 g, 26.5 mmol) in 70 ml of methanol during boiling. After holding for 1.25 h, the solvent was removed. The residue was extracted with dichloromethane, and the solvent was removed under vacuum. Product 1.9 was isolated in the form of a light-brown powder after recrystallization from ethanol. Yield—4.78 g (80%).

Step 4. Compound 1.9 (1.50 g, 6.60 mmol) was added to a solution of LiOH×H₂O (306 mg, 7.20 mmol) in 30 ml of a water-THF mixture. The reaction mass was stirred for 3 hours, and the solvent was removed. 5 ml of MTBE were added to the mixture. The layers were separated, the aqueous layer was acidified to pH 2-3 by adding 0.75 ml of concentrated HCl. The precipitate was filtered off, washed with water, dried in air, and dissolved in 14 ml of anhydrous dichloromethane under nitrogen atmosphere, followed by adding SOCl₂ (1.40 ml, 19.3 mmol) and 50 μl of DMF. The reaction mass was stirred for 3 hours while boiling. Next, the solvent was removed until the mixture was dry, 5 ml of toluene were added and the mixture was re-concentrated under vacuum. Product 7.3 was obtained in the form of a solid brown mass. Yield—1.36 g (90%).

Step 5. A solution of compound 7.3 (580 mg, 2.50 mmol) in 10 ml of dichloromethane was added dropwise to a solution of 1-isopropylpiperazine (390 mg, 3.00 mmol) and DIPEA (510 mg, 5.00 mmol) in 20 ml of dichloromethane, while stirring and cooling in an ice bath under a nitrogen atmosphere. Stirring was continued at 20° C. for 3 h, followed by washing the reaction mass with water. Next, the organic layer was mixed with Na₂SO₄ and activated carbon for 15 min, filtered through a celite layer, and subject to solvent removal. Product 7.2 was obtained in the form of an orange viscous liquid. Yield—800 mg (99%).

Compounds 12.2, 13.2, and 17.2 were obtained in a similar manner from the corresponding starting reagents, shown in Table 1.

TABLE 1

| | Starting | |
|---|---|---|
| Starting reagent | reagent | Compound |
| 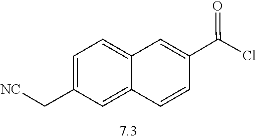 [SMILES: N#CCc1ccc2cc(C(=O)Cl)ccc2c1] | 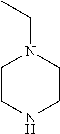 | 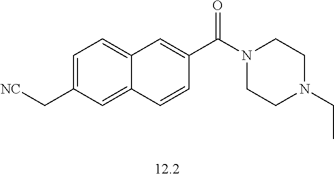 [SMILES: CCN1CCN(C(=O)c2ccc3cc(CC#N)ccc3c2)CC1] |
| | 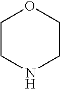 | 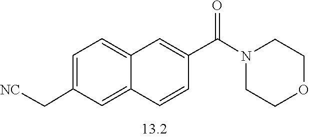 [SMILES: N#CCc1ccc2cc(C(=O)N3CCOCC3)ccc2c1] |
| | 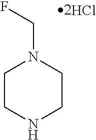 | 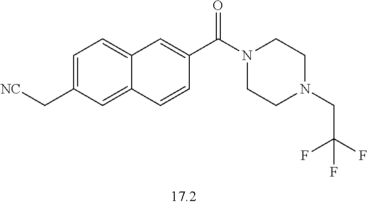 [SMILES: N#CCc1ccc2cc(C(=O)N3CCN(CC(F)(F)F)CC3)ccc2c1] |

1-(2,2,2-trifluoroethyl)piperazine dihydrochloride can be obtained according to the procedure described in the Journal of Medicinal Chemistry 2007, 50(15), 3528.

### Example 2. A Method of Obtaining Compound 1

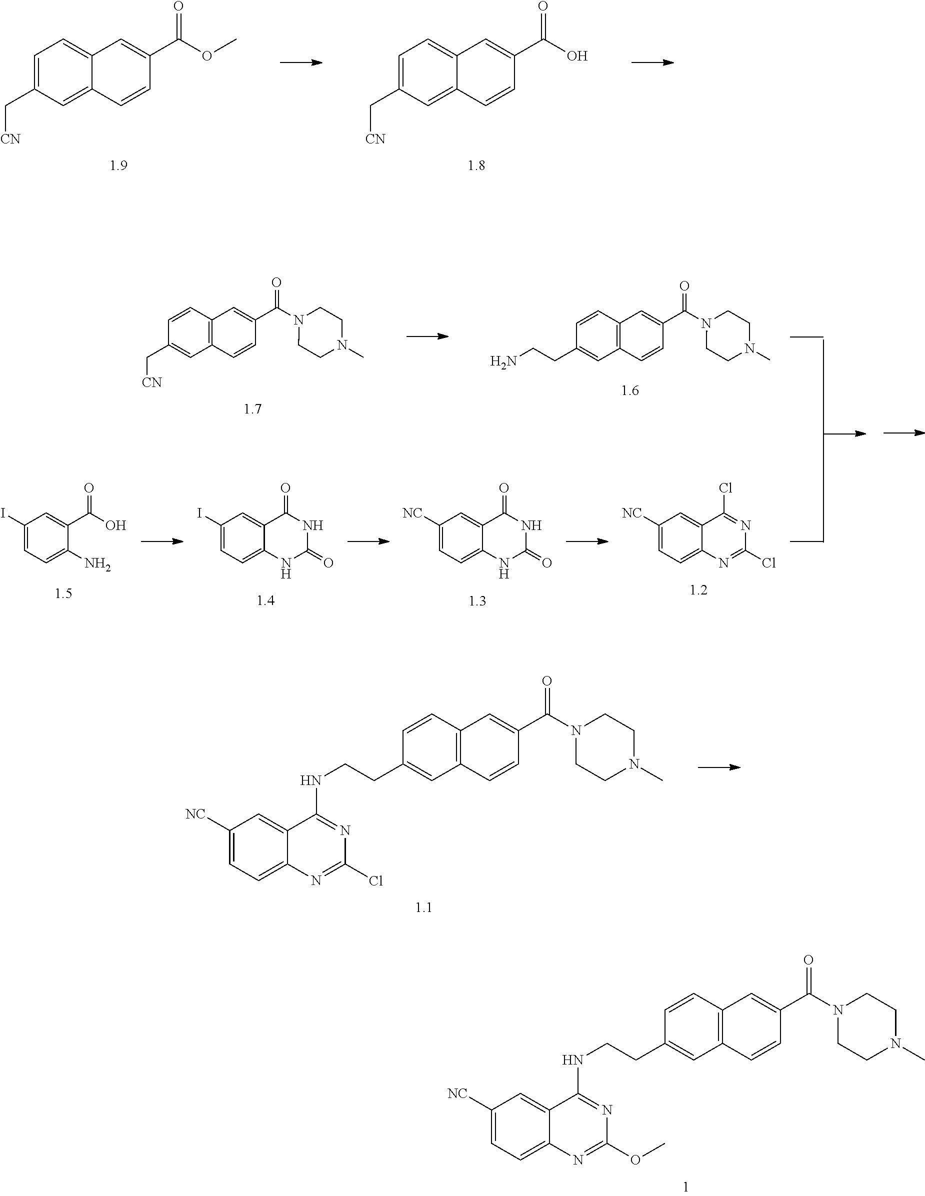

Step 1. Compound 1.9 (4.78 g, 21.2 mmol) was added to a solution of LiOH (559 mg, 23.3 mmol) in 64 ml of water and 52 ml of THF over the period of 1.5 h. After stirring for 4 h at 30° C., THF was removed. The aqueous solution was washed with MTBE and acidified with 1M of HCl to pH 2. The precipitate of product 1.8 was filtered, washed with water, and dried under vacuum to a constant weight. Yield—4.39 g (98%).

Step 2. SOCl₂ (7.42 g, 62.4 mmol) and DMF (15 mg, 0.208 mmol) were added to compound 1.8 (4.39 g, 20.8 mmol) suspended in 44 m of anhydrous dichloromethane. The reaction mixture was stirred for 4 hours while boiling. The solvent was then removed, and the residue was dissolved in 45 ml of anhydrous dichloromethane. The resulting solution was added dropwise to a solution of triethylamine (2.35 g, 22.9 mmol) and N-methylpiperazine (2.19 g, 21.84 mmol) in 45 ml of anhydrous dichloromethane at 5° C. After 30 minutes, the resulting mixture was warmed to room temperature, washed with water, dried over Na₂SO₄, and the solvent was removed. Product 1.7 was isolated in the form of a brown oily liquid. Yield—5.80 g (95%).

Step 3. 50 ml of a 25% ammonia solution were added to a solution of compound 1.7 (5.80 g, 19.8 mmol) in 50 ml of methanol, followed by adding a suspension of freshly prepared Raney Ni catalyst (2.90 g, 49.0 mmol) in 20 ml of methanol. After holding for 4 h in an autoclave filled with hydrogen at a pressure of 10 atm, the reaction mixture was filtered through a celite, and the filtrate was removed under vacuum. The residue was extracted with dichloromethane, and the solvent was removed under vacuum. Product 1.6 was isolated in the form of a brown oily liquid. Yield—5.58 g (95%).

Compounds 7.1, 12.1, and 17.1 were obtained in a similar manner from the corresponding starting reagents shown in Table 2.

TABLE 2

| Starting reagent | Compound |
|---|---|
| 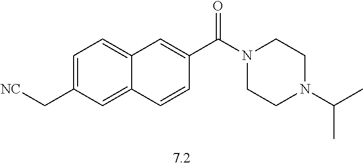 [SMILES: CC(C)N1CCN(C(=O)c2ccc3cc(CC#N)ccc3c2)CC1] | 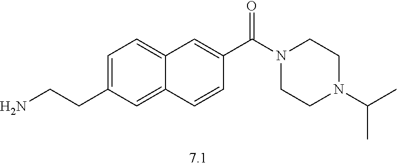 [SMILES: CC(C)N1CCN(C(=O)c2ccc3cc(CCN)ccc3c2)CC1] |
| 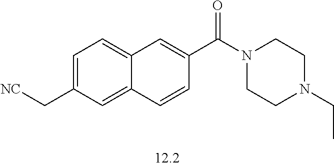 [SMILES: CCN1CCN(C(=O)c2ccc3cc(CC#N)ccc3c2)CC1] | 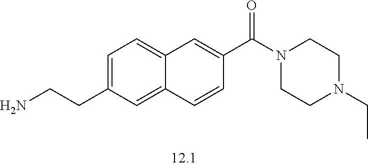 [SMILES: CCN1CCN(C(=O)c2ccc3cc(CCN)ccc3c2)CC1] |
| 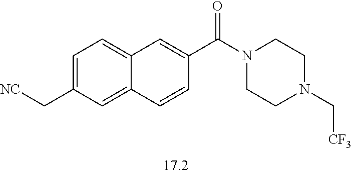 [SMILES: N#CCc1ccc2cc(C(=O)N3CCN(CC(F)(F)F)CC3)ccc2c1] | 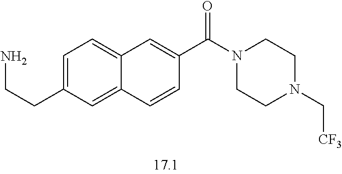 [SMILES: NCCc1ccc2cc(C(=O)N3CCN(CC(F)(F)F)CC3)ccc2c1] |

Step 4. Urea (22.8 g, 380 mmol) was added to compound 1.5 (10.0 g, 38 mmol) suspended in 70 ml of DMAA. After holding for 3 h at 160° C., the reaction mixture was cooled to room temperature and poured into 210 ml of water. The precipitate of product 1.4 was filtered, washed with water, and dried under vacuum to constant weight. Yield—8.30 g (76%).

Step 5. Zn(CN)₂ (2.77 g, 23.7 mmol) and Pd(PPh₃)₄ (335 mg, 0.29 mmol, 0.01 eq.) were added to compound 1.4 (8.30 g, 28.9 mmol) suspended in 30 ml of DMF. Nitrogen was passed through the reaction mass for 15 minutes, followed by stirring for 2 h at 120° C. The hot reaction mixture was filtered through celite and washed with DMF. The filtrate was cooled to 5° C. The precipitate of product 1.3 was filtered, washed with acetonitrile and water, and dried under vacuum to constant weight. Yield—2.90 g (54%).

Step 6. DIPEA (5.84 g, 45.2 mmol) was added to compound 1.3 (2.90 g, 15.6 mmol) suspended in 5.8 ml of toluene. POCl₃ (14.35 g, 93.6 mmol) was added to the mixture at a temperature of 0° C. After holding for 30 min at 0° C. and 3 hours at 90° C., the mixture was cooled to room temperature and poured into ice. The resulting mixture was adjusted with the NaHCO₃ solution to pH 8. The precipitate was filtered and washed with water. Product 1.2 was isolated by silica gel column chromatography using hexane dichloromethane as eluent (ratio—1:9) in the form of a brown powder. Yield—2.70 g (78%).

Step 7. Triethylamine (3.70 g, 36.6 mmol) and compound 1.2 (2.70 g, 12.2 mmol) were added to a solution of compound 1.6 (4.00 g, 13.5 mmol) in 60 ml of acetonitrile at 0° C. After holding at room temperature for 2 h, a precipitate of product 1.1 was filtered, washed with acetonitrile and water, and dried under vacuum to constant weight. Yield—4.86 g (82%).

Step 8. A suspension of pyrimidine 1.1 (100 mg, 0.206 mmol) and sodium methylate (13 mg, 0.247 mmol) in 10 ml of the methanol-NMP mixture (ratio—2:1) was stirred under pressure in a closed vessel at 100° C. for 15 h. After removal of the volatile components under reduced pressure, product 1 was isolated in the form of a white powder using preparative chromatography (Method A). Yield—31 mg (31%).

### Example 3. A Method of Obtaining Compound 2

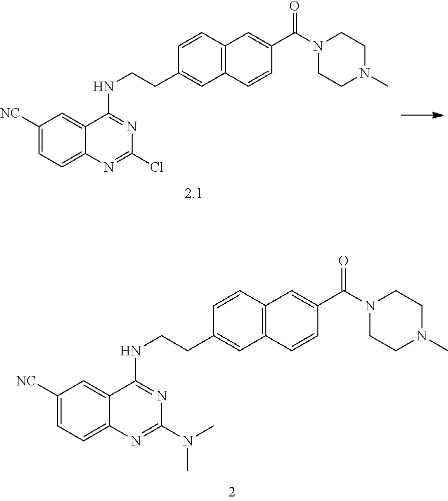 [SMILES: CN1CCN(C(=O)c2ccc3cc(CCNc4nc(Cl)nc5ccc(C#N)cc45)ccc3c2)CC1 ; CN1CCN(C(=O)c2ccc3cc(CCNc4nc(N(C)C)nc5ccc(C#N)cc45)ccc3c2)CC1]

A suspension of pyrimidine 2.1 (100 mg, 0.206 mmol), dimethylamine (31 μl, 0.247 mmol, 40% aqueous solution), and triethylamine (30 μl, 0.216 mmol) in 3 ml of DMF was stirred under pressure in a closed vessel at 120° C. for 2 hours. After concentration of the reaction mixture, the product was isolated in the form of a beige powder using preparative chromatography (Method A). Yield—50 mg (52%).

### Example 4. A Method of Obtaining Compound 3

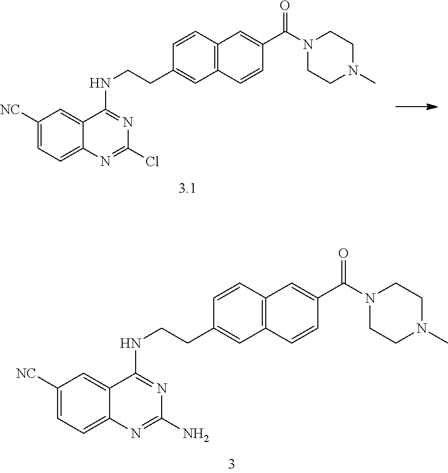 [SMILES: CN1CCN(C(=O)c2ccc3cc(CCNc4nc(Cl)nc5ccc(C#N)cc45)ccc3c2)CC1 ; CN1CCN(C(=O)c2ccc3cc(CCNc4nc(N)nc5ccc(C#N)cc45)ccc3c2)CC1]

A suspension of pyrimidine 3.1 (100 mg, 0.206 mmol) in 200 μl of 25% aqueous ammonia was stirred under pressure in a closed vessel at 150° C. for 8 hours. After concentration of the reaction mixture, the product was isolated in the form of a white powder using preparative chromatography (Method B). Yield—55 mg (54%).

### Example 5. A Method of Obtaining Compound 4

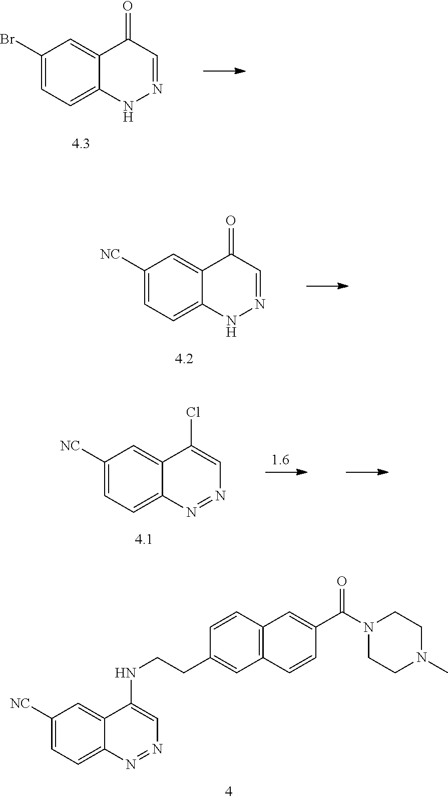 [SMILES: O=c1cn[nH]c2ccc(Br)cc12 ; N#Cc1ccc2[nH]ncc(=O)c2c1 ; N#Cc1ccc2nncc(Cl)c2c1 ; CN1CCN(C(=O)c2ccc3cc(CCNc4cnnc5ccc(C#N)cc45)ccc3c2)CC1]

Step 1. Compound 4.2 was prepared similarly to compound 1.3 (Example 2, step 5) using compound 4.3 (prepared according to the procedure described in Bioorganic & Medicinal Chemistry 2006, 14 (20), 6832) instead of compound 1.4.

Step 2. Compound 4.1 was prepared similarly to compound 1.2 (Example 2, step 6) using compound 4.2 instead of compound 1.3.

Step 3. Compound 4.1 (130 mg, 0.686 mmol) was added to a solution of amine 1.6 (212 mg, 0.713 mmol) and triethylamine (287 μl, 2.06 mmol) in 5 ml of acetonitrile at 10° C. in a flow of nitrogen. After 10 minutes, the temperature was raised to 60° C., and the mixture was held under heating for 8 hours. The suspension was then filtered, and the resulting precipitate was washed with acetonitrile. Product 4 in the form of a yellow powder was isolated by silica gel column chromatography using dichloromethane-methanol as eluent (ratio—95:5). Yield—128 mg (41%).

### Example 6. A Method of Obtaining Compounds 5, 5a, 7, 12, 13, 17

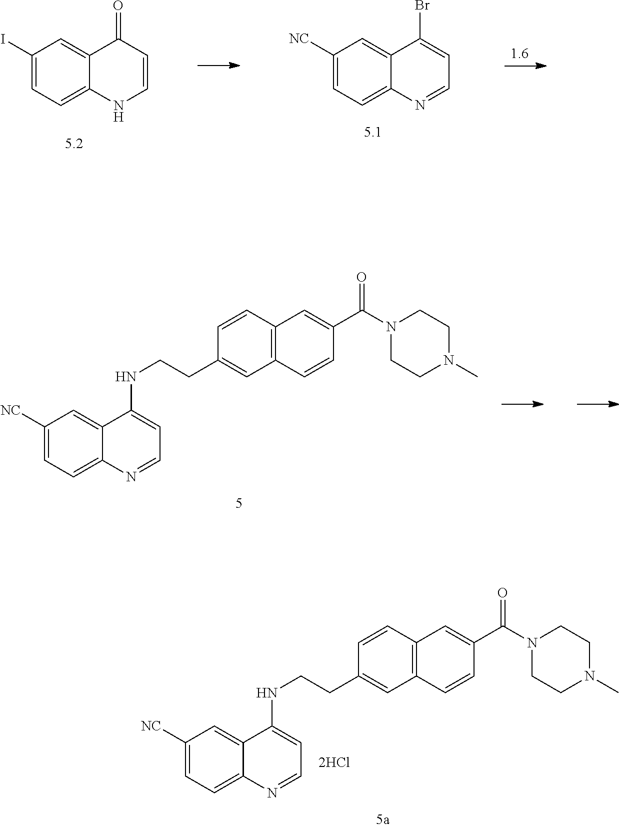 [SMILES: O=c1cc[nH]c2ccc(I)cc12 ; N#Cc1ccc2nccc(Br)c2c1 ; CN1CCN(C(=O)c2ccc3cc(CCNc4ccnc5ccc(C#N)cc45)ccc3c2)CC1 ; CN1CCN(C(=O)c2ccc3cc(CCNc4ccnc5ccc(C#N)cc45)ccc3c2)CC1]

Step 1. Nitrogen was passed through a suspension of 6-iodo-4-hydroxyquinoline (8.00 g, 29.5 mmol) in 60 ml of DMF for 10 min, followed by the addition of Zn(CN)₂ (4.16 g, 35.4 mmol) and Pd(PPh₃)₄ (1.71 g, 1.48 mmol, 0.05 eq.). The mixture was stirred under a nitrogen atmosphere at 100° C. for 2 hours. Then the reaction mixture was brought to room temperature and, while cooling in an ice bath, PBr₃ (3 ml, 32.5 mmol) was added dropwise. Once the addition was completed, the mixture was held for 10 min at 0° C., and then stirred for 1 h at room temperature. The mixture was neutralized with saturated Na₂CO₃ solution, and 10 the product was extracted with dichloromethane, dried over Na₂SO₄, and concentrated in vacuum. Product 5.1 was isolated by silica gel column chromatography using hexane-ethyl acetate-dichloromethane as eluent (ratio—4:1:1) in the form of a white powder. Yield—4.28 g (62%).

Step 2. Nitrogen was passed for 10 min through a suspension of compound 1.6 (842 mg, 2.83 mmol), compound 5.1 (600 mg, 2.57 mmol), and Cs₂CO₃ (1.68 g, 5.15 mmol) in 20 ml of dioxane. Then, BINAP (321 mg, 0.516 mmol, 0.20 eq.) and palladium (II) acetate (58 mg, 0.259 mmol, 0.10 eq.) were added and stirred under an inert atmosphere for 3 h at 100° C. After concentrating the reaction mixture, product 5 was isolated in the form of a pale-yellow powder by silica gel column chromatography using dichloromethane-methanol as eluent (ratio—95:5). Yield—640 mg (55%).

Compounds 7, 12, 13, and 17 were obtained in a similar manner from the corresponding starting reagents shown in Table 3.

TABLE 3

| | Starting | |
|---|---|---|
| Starting reagent | reagent | Compounds |
| 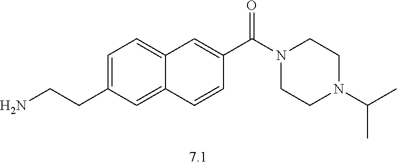 [SMILES: CC(C)N1CCN(C(=O)c2ccc3cc(CCN)ccc3c2)CC1] | 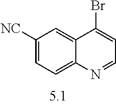 [SMILES: N#Cc1ccc2nccc(Br)c2c1] | 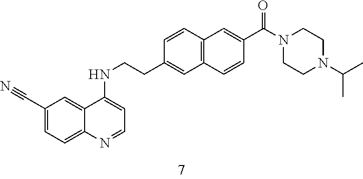 [SMILES: CC(C)N1CCN(C(=O)c2ccc3cc(CCNc4ccnc5ccc(C#N)cc45)ccc3c2)CC1] |
| 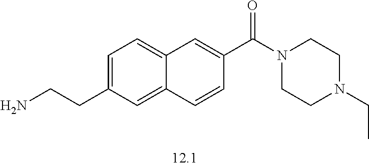 [SMILES: CCN1CCN(C(=O)c2ccc3cc(CCN)ccc3c2)CC1] | | 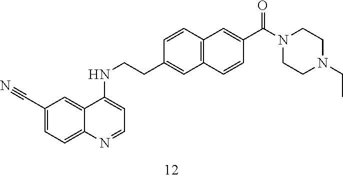 [SMILES: CCN1CCN(C(=O)c2ccc3cc(CCNc4ccnc5ccc(C#N)cc45)ccc3c2)CC1] |
| | | The product was additionally |
| | | purified by using a preparative |
| | | chromatography (Method B). |
| 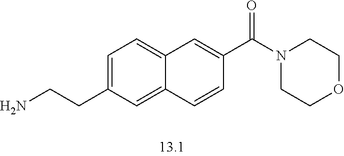 [SMILES: NCCc1ccc2cc(C(=O)N3CCOCC3)ccc2c1] | | 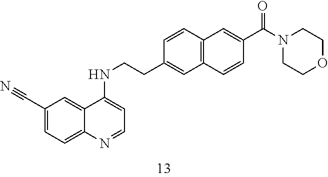 [SMILES: N#Cc1ccc2nccc(NCCc3ccc4cc(C(=O)N5CCOCC5)ccc4c3)c2c1] |
| 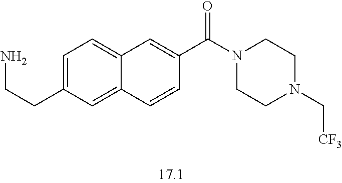 [SMILES: NCCc1ccc2cc(C(=O)N3CCN(CC(F)(F)F)CC3)ccc2c1] | | 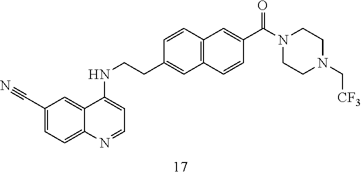 [SMILES: N#Cc1ccc2nccc(NCCc3ccc4cc(C(=O)N5CCN(CC(F)(F)F)CC5)ccc4c3)c2c1] |

Step 3. A 4M solution of HCl in diethyl ether (605 μl, 2.42 mmol) was added dropwise to compound 5 (495 mg, 1.10 mmol) suspended in 50 ml of methanol. After 1 h, the solvents were removed, 30 ml of diethyl ether were added, and the resulting yellow precipitate of compound 5a was filtered off. Yield—422 mg (73%).

### Example 7. A Method of Obtaining Compound 6

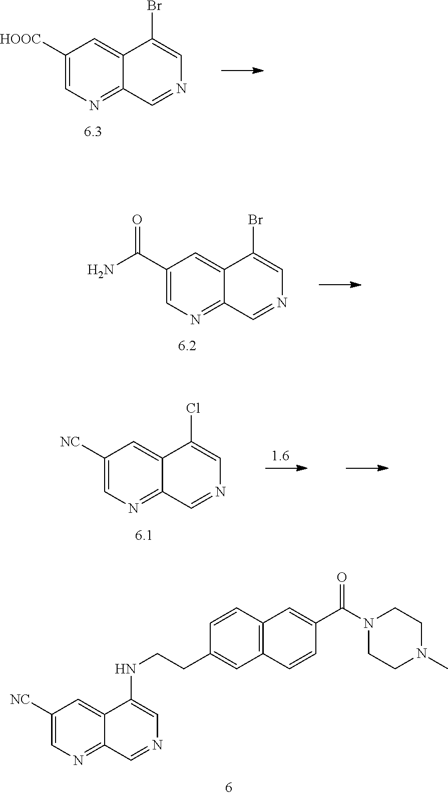 [SMILES: O=C(O)c1cnc2cncc(Br)c2c1 ; NC(=O)c1cnc2cncc(Br)c2c1 ; N#Cc1cnc2cncc(Cl)c2c1 ; CN1CCN(C(=O)c2ccc3cc(CCNc4cncc5ncc(C#N)cc45)ccc3c2)CC1]

Step 1. 5 ml of SOCl₂ and one drop of DMF were added to 5-bromo-1,7-naphthyridine-3-carboxylic acid 6.3 (500 mg, 1.97 mmol) (obtained according to the procedure described in WO 2015/014768). The reaction mixture was stirred while boiling for 5 hours, after which the volatiles were removed under reduced pressure. 5 ml of MTBE were added to the residue, after which the latter was concentrated and dried under vacuum in a rotary evaporator. A suspension of the obtained powder in 10 ml of dichloromethane was added to 20 ml of a 4M solution of NH₃ in methanol at 0° C. The reaction mass was stirred for 8 hours, after which the volatile components were removed under reduced pressure. As a result of the reaction, product 6.2 was isolated as a brown powder and used during the next step without additional purification. Yield—450 mg (90%).

Step 2. Pyridine (190 μl, 2.38 mmol) and trifluoroacetic anhydride (180 μl, 1.42 mmol) were added to a solution of compound 6.2 (300 mg, 1.19 mmol) in 6 ml of 1,4-dioxane at 0° C. The reaction mass was stirred at 40° C. for 1.5 hours. Next, the reaction mass was poured into 40 ml of water and extracted with ethyl acetate. The organic layer was separated, dried with Na₂SO₄ and concentrated under vacuum. Product 6.1 in the form of a white powder was isolated by silica gel column chromatography using dichloromethane-methanol as eluent (97:3). Yield—180 mg (65%).

Step 3. Compound 6 was obtained similarly to compound 5 (Example 6, Step 2), using compound 6.1 instead of compound 5.1.

### Example 8. A Method of Obtaining Compound 8

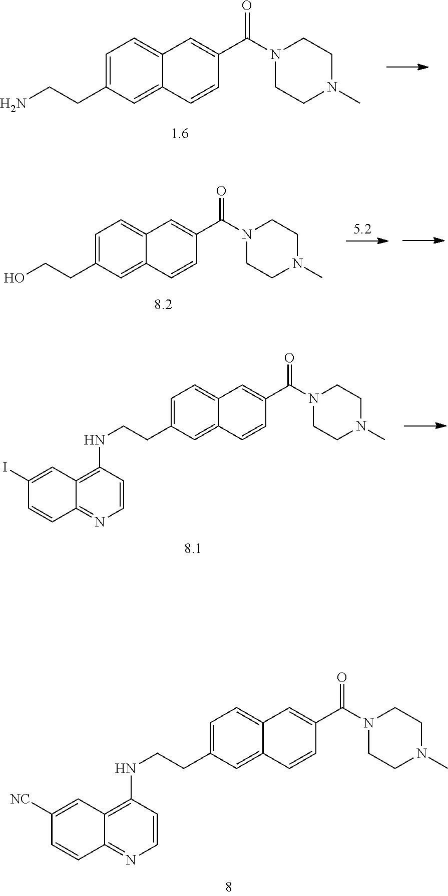 [SMILES: CN1CCN(C(=O)c2ccc3cc(CCN)ccc3c2)CC1 ; CN1CCN(C(=O)c2ccc3cc(CCO)ccc3c2)CC1 ; CN1CCN(C(=O)c2ccc3cc(CCNc4ccnc5ccc(I)cc45)ccc3c2)CC1 ; CN1CCN(C(=O)c2ccc3cc(CCNc4ccnc5ccc(C#N)cc45)ccc3c2)CC1]

Step 1. A solution of sodium nitrite (230 mg, 330 mmol) in 2.3 ml of water was added dropwise to a solution of compound 1.6 (1.00 g, 3.36 mmol) in a mixture of 10 ml of water and 10 ml of glacial acetic acid at 0° C. The solution was kept at this temperature for 30 min, and then at 80° C. for 1 h. The reaction mass was neutralized with a 25% aqueous NH₃ solution to pH 9 and extracted with ethyl acetate-methanol (9:1). Next, the solvent was removed, and the residue was dissolved in 10 ml of THF. 33H (38 mg, 1.6 mmol) and 10 ml of water were added to the resulting mixture. After holding for 3 hours at room temperature, 20 ml of ethyl acetate were added to the mixture. The reaction mixture was extracted with 1-butanol, and the solvent was removed under vacuum. Product 8.2 was isolated in the form of a yellow oily residue by silica gel column chromatography using dichloromethane-methanol as eluent (ratio—94:6). Yield—650 mg (68%).

Step 2. Triphenylphosphine (131 mg, 0.50 mmol) and 4-hydroxy-6-iodoquinoline 5.2 (134 mg, 0.50 mmol) were added to a solution of compound 8.2 (131 mg, 4.90 mmol) in 10 ml of anhydrous THF. Then, DIAD was added dropwise (101 mg, 0.50 mmol) at 0° C. The reaction mass was kept at room temperature for 16 hours. The solvent was removed under vacuum, and product 8.1 was isolated in the form of a yellow oily residue by silica gel column chromatography using a dichloromethane-methanol as eluent (ratio—98:2-92:8). Yield—40 mg (22%).

Step 3. Compound 8 was prepared similarly to compound 1.3 (Example 2, step 5) using compound 8.1 instead of compound 1.4. The product was further purified by preparative chromatography (Method B).

### Example 9. A Method of Obtaining Compound 9

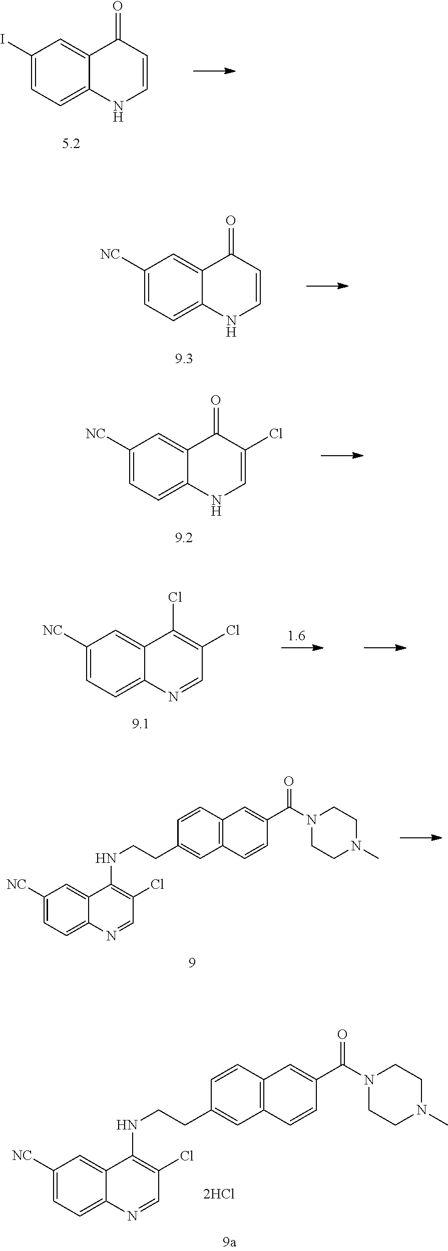 [SMILES: O=c1cc[nH]c2ccc(I)cc12 ; N#Cc1ccc2[nH]ccc(=O)c2c1 ; N#Cc1ccc2[nH]cc(Cl)c(=O)c2c1 ; N#Cc1ccc2ncc(Cl)c(Cl)c2c1 ; CN1CCN(C(=O)c2ccc3cc(CCNc4c(Cl)cnc5ccc(C#N)cc45)ccc3c2)CC1 ; CN1CCN(C(=O)c2ccc3cc(CCNc4c(Cl)cnc5ccc(C#N)cc45)ccc3c2)CC1]

Step 1. Compound 9.3 was obtained similarly to compound 1.3 (Example 2, Step 5) using compound 5.2 instead of compound 1.4.

Step 2. N-chlorosuccinimide (428 mg, 3.17 mmol) was added to a solution of nitrile 9.3 (500 mg, 2.64 mmol) in glacial acetic acid at 50° C. The resulting mixture was stirred at 65° C. for 1 h, cooled to 2° C., and dried under vacuum after removing the precipitate. Product 9.2 was used during the next step without additional purification. Yield—480 mg (89%).

Step 3. Compound 9.2 (430 mg, 1.89 mmol) in 5 ml of POCl₃ was stirred while boiling for 1 h, then the reaction mixture was poured into ice, neutralized with a saturated NaHCO₃ solution, and extracted with ethyl acetate. The combined organic fractions were concentrated under vacuum, and product 9.1 was isolated by silica gel column chromatography using ethyl acetate-hexane as eluent (ratio—50:50). Yield—270 mg (64%).

Step 4. Compound 9 was prepared similarly to compound 4 (Example 5, Step 3) using compound 9.1 instead of compound 4.1.

Step 5. Compound 9a was prepared similarly to compound 5a (Example 6, Step 3) using compound 9 instead of compound 5. The product was further purified by preparative chromatography (Method D).

### Example 10. A Method of Obtaining Compound 11

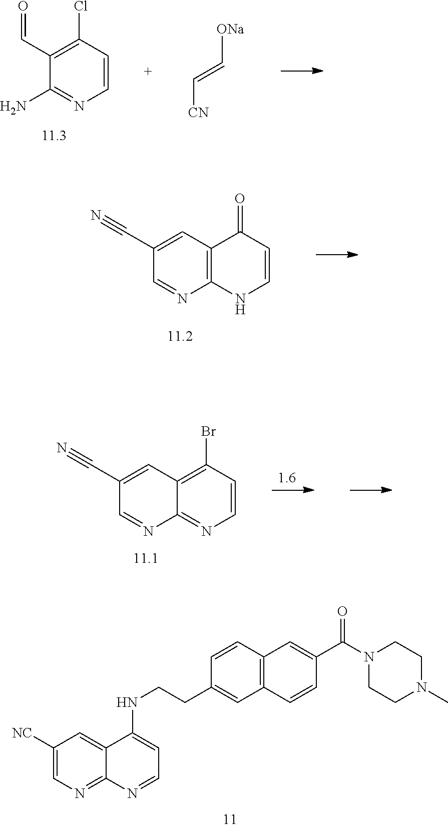

Step 1. 2-Amino-4-chloronicotinaldehyde 11.3 (313 mg, 2.00 mmol) (obtained according to the procedure described in the Journal of Medicinal Chemistry 2010, 53 (8), 3330), sodium 2-cyanethenolate (520 mg, 5.60 mmol) (obtained according to the procedure described in the Journal of the Chemical Society, Perkin Transactions 1: Organic and Bio-Organic Chemistry (1972-1999), 1986, 5, 747), and 8 ml of acetic acid were stirred at 70° C. for 5 hours, followed by cooling the reaction mass down and concentrating it under vacuum. Product 11.2 was isolated in the form of a solid gray mass by column chromatography on silica gel with ethyl acetate and then ethyl acetate-methanol used as eluents (ratio—9:1). Yield—77 mg (20%).

Step 2. PBr₃ (120 mg, 0.45 mmol) was added to a solution 11.2 (73 mg, 0.43 mmol) in 1 ml of DMF. The reaction mass was stirred at room temperature in the nitrogen atmosphere for 2 hours, and then concentrated under vacuum. Product 11.1 was isolated in the form of a solid gray mass by column chromatography on silica gel using ethyl acetate-hexane as eluent (ratio—1:1). Yield—90 mg (70%).

Step 3. Compound 11 was obtained similarly to compound 5 (Example 6, Step 2), using compound 11.1 instead of compound 5.1.

### Example 11. A Method of Obtaining Compound 18

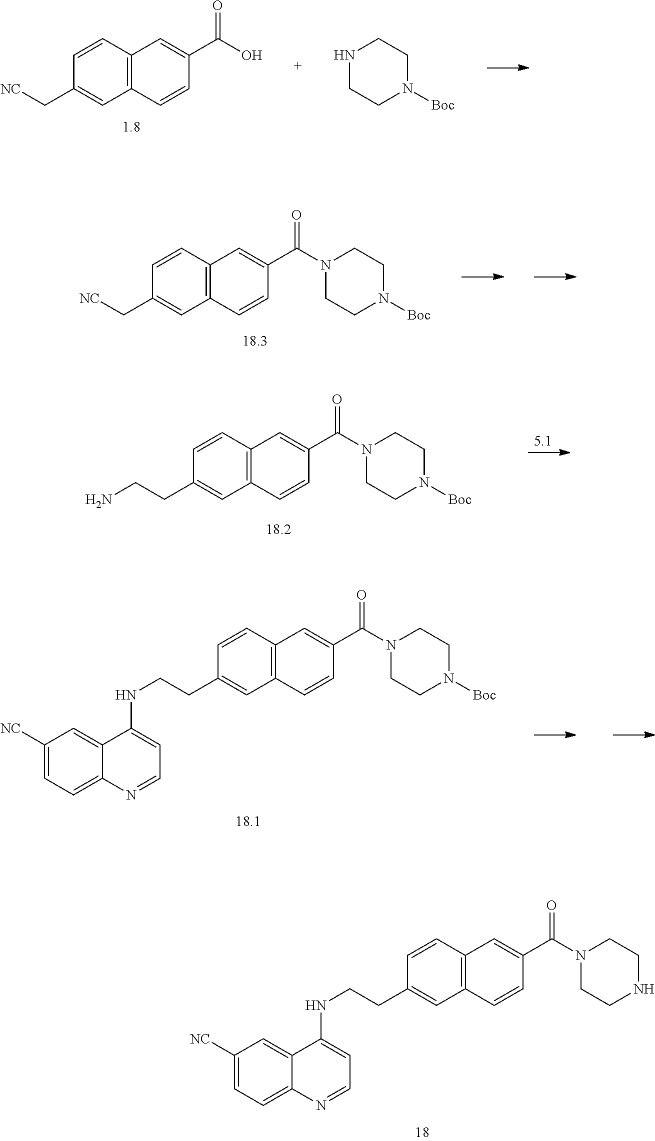 [SMILES: N#CCc1ccc2cc(C(=O)O)ccc2c1 ; CC(C)(C)OC(=O)N1CCNCC1 ; CC(C)(C)OC(=O)N1CCN(C(=O)c2ccc3cc(CC#N)ccc3c2)CC1 ; CC(C)(C)OC(=O)N1CCN(C(=O)c2ccc3cc(CCN)ccc3c2)CC1 ; CC(C)(C)OC(=O)N1CCN(C(=O)c2ccc3cc(CCNc4ccnc5ccc(C#N)cc45)ccc3c2)CC1 ; N#Cc1ccc2nccc(NCCc3ccc4cc(C(=O)N5CCNCC5)ccc4c3)c2c1]

Step 1. A mixture of acid 1.8 (1.00 g, 4.74 mmol) and N-Boc-piperazine (971 mg, 5.21 mmol) was dissolved in 10 ml of DMF, followed by adding CDI (922 mg, 5.69 mmol) in portions, while cooling in an ice bath. The reaction mixture was stirred at room temperature for 18 hours, after which 100 ml of water was added. The resulting precipitate was filtered and washed with water. Product 18.3 was isolated in the form of a light yellow powder by silica gel column chromatography using dichloromethane-methanol as eluent (50.1). Yield—281 mg (16%).

Step 2. Compound 18.2 was obtained similarly to compound 1.6 (Example 2, Step 3) using compound 18.3 instead of compound 1.7.

Step 3. Compound 18.1 was obtained similarly to compound 5 (Example 6, Step 2) using compound 18.2 instead of compound 1.6.

Step 4. 1.5 ml of a 4M solution of HCl in dioxane were added dropwise to a solution of compound 18.1 (71 mg, 0.133 mmol) in 3 ml of dichloromethane, while cooling in a water bath. After 16 hours, the precipitate was filtered and washed with dichloromethane. Yield—48 mg (79%). Product 18 of the reaction was further purified by preparative chromatography (Method B).

### Example 12. A Method of Obtaining Compound 19

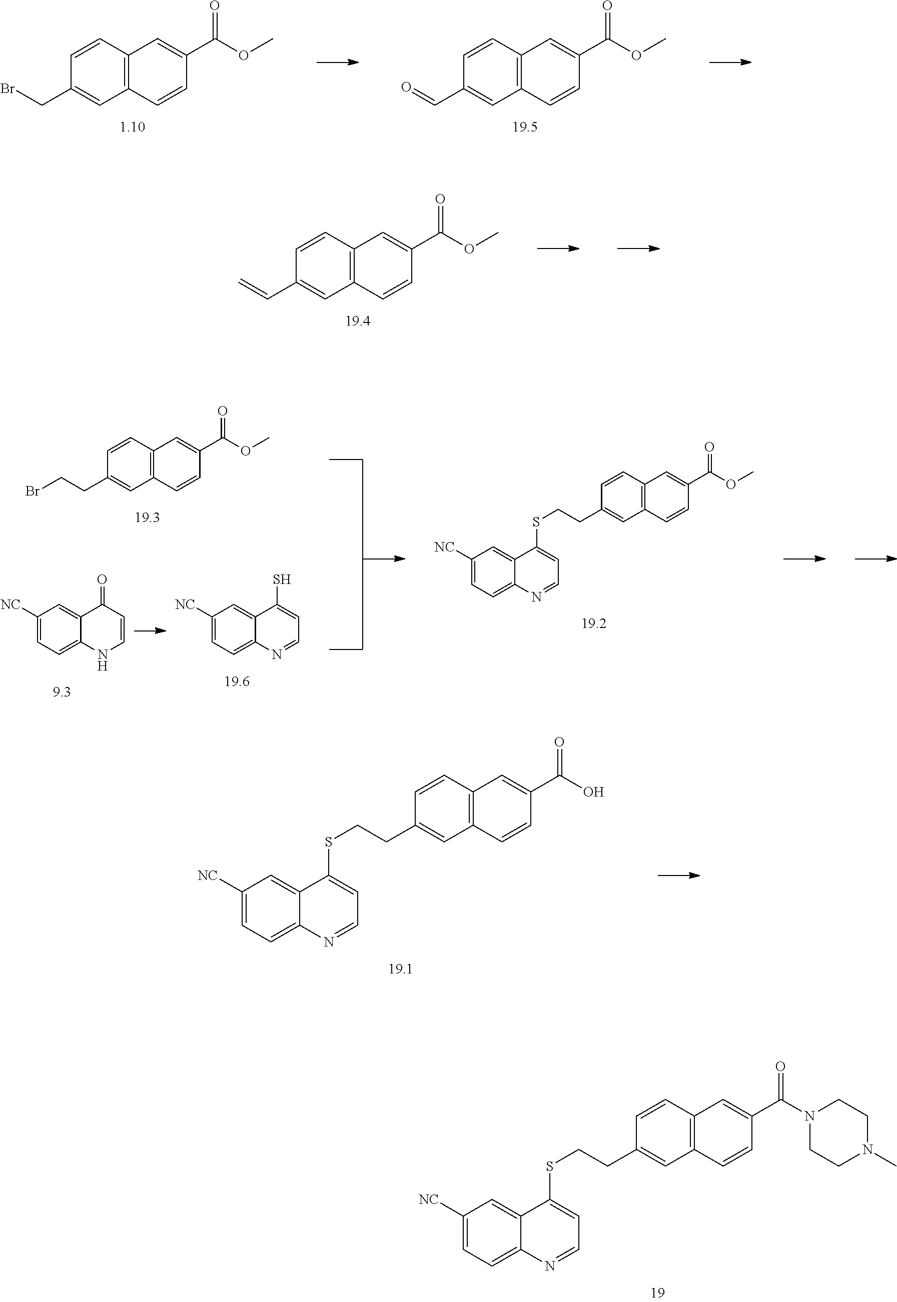

Step 1. Urotropine (1.58 g, 11.2 mmol) was added to compound 1.10 (3.00 g, 10.2 mmol) suspended in 20 ml of an ethanol-water mixture (ratio—1:1) at room temperature. The reaction mixture was stirred while boiling for 9 hours. Then, 10 ml of concentrated HCl were added at room temperature, and the mixture was stirred for 18 hours. 30 ml of water were then added to the reaction mixture. A precipitate of product 19.5 was filtered, washed with water and hexane, and dried in air. Yield—1.38 g (43%).

Step 2. A suspension of methyltriphenylphosphonium iodide (5.36 g, 12.6 mmol) in 50 ml of THF was cooled to −60° C., followed by gradual addition of a 2.5 M butyl-lithium solution in hexane (5.60 ml, 12.6 mmol). The reaction mixture was heated to room temperature over a period of 1 h. Next, compound 19.5 (2.50 g, 10.5 mmol) in 35 ml of THF was added at −20° C. and stirred at room temperature for 1 h. The reaction mixture was extracted with ethyl acetate. The combined organic fractions were concentrated in vacuum. Product 19.4 was isolated by silica gel column chromatography using hexane-ethyl acetate as eluent (ratio—97:3). Yield—2.07 g (93%).

Step 3. A mixture of compound 19.4 (2.00 g, 8.48 mmol) and AIBN (280 mg, 1.69 mmol) in carbon tetrachloride was brought to a boil, followed by adding 30% HBr solution in acetic acid (5 ml, 25.4 mmol) and stirring while boiling for 6 hours. The reaction mixture was extracted with dichloromethane. The combined organic fractions were concentrated in vacuum. Product 19.3 was isolated by silica gel column chromatography using hexane-ethyl acetate as eluent (ratio—95:5). Yield—2.0 g (80%).

Step 4. A mixture of compound 9.3 (400 mg, 2.11 mmol) and Lawesson's reagent (864 mg, 2.11 mmol) in 7 ml of pyridine was stirred at room temperature for 1 h. The reaction mixture was extracted with ethyl acetate and concentrated in vacuum. Product 19.6 was isolated by silica gel column chromatography using dichloromethane as eluent. Yield—350 mg (88%).

Step 5. Argon was passed through the mixture of compounds 19.3 (672 mg, 2.30 mmol), 19.6 (385 mg, 2.07 mmol) and K₂CO₃ (643 mg, 4.60 mmol) in 10 ml of DMSO for 10 min, after which the mixture was stirred for 10 h at room temperature. The reaction mixture was extracted with ethyl acetate and concentrated in vacuum. Product 19.2 was isolated by silica gel column chromatography using hexane-ethyl acetate as eluent (ratio—85:15). Yield—412 mg (50%).

Step 6. Compound 19.1 was obtained similarly to compound 1.8 (Example 2, Step 1), using compound 19.2 instead of compound 1.9.

Step 7. EDC×HCl (65 mg, 0.34 mmol) was added to the mixture of compound 19.1 (100 mg, 0.26 mmol), N-methylpiperazine (32 μl, 0.29 mmol), and DMAP (3 mg, 0.03 mmol) in 5 ml of dichloromethane. The reaction mixture was stirred under nitrogen for 8 hours. The reaction mixture was extracted with dichloromethane and concentrated in vacuum. Product 19 was isolated by silica gel column chromatography using dichloromethane-methanol as eluent (ratio—95:5). Yield—45 mg (37%). The product was further purified by preparative chromatography (Method B).

### Example 13. Analysis of the Obtained Compounds

The purity and structure of the obtained compounds were confirmed by the chromatography-mass spectrometry (LC/MS) and spectroscopy (1H NMR) (Table 6).

### Equipment Data:

TABLE 4
Chromatography-mass spectrometry

| | Manufacturer, |
|---|---|
| Name | country |
| Agilent Triple Quad Liquid Chromatograph | Agilent, USA |
| LC/MS: | |
| Agilent 1200 auto-sampler | |
| Agilent 1200 column thermostat | |
| Agilent 1200 degasser | |
| Agilent 1200 auto-sampler thermostat | |
| Agilent 6410 QQQ MS-detector | |
| Agilent 1200 UV-detector | |
| Agilent 1200 pump | |

TABLE 5
NMR spectrometer

| | Manufacturer, | Model, main |
|---|---|---|
| Name | country | characteristics |
| NMR spectrometer | Germany | AVANCE III, 400 MHz |

TABLE 6
Analytical data for compounds used as examples

| | ESI-MS · | NMR ¹H, |
|---|---|---|
| | [M + 2H]⁺/ | DMSO-d6, |
| Number | 2, [M + H]⁺ | δ, md |
| 1 | 241.2, | 8.76 (t, J = 5.4 Hz, 1H), 8.73 (d, J = 1.6 |
| | 481.3 | Hz, 1H), 8.16 (s, 1H), 7.93-7.88 |
| | | (m, 4H), 7.83 (s, 1H), 7.58 (d, J = 8.7 |
| | | Hz, 1H), 7.53 (dd, J = 8.4, 1.5 Hz, 1H), |
| | | 7.46 (dd, J = 8.4, 1.6 Hz, 1H), 3.94 (s, |
| | | 1H), 3.89-3.80 (m, 2H), 3.79-3.23 |
| | | (signal in water, 4H), 3.22-3.11 (m, |
| | | 2H), 2.45-2.25 (m, 4H), 2.21 (s, 3H). |
| 2 | 247.7, | 8.50 (d, J = 1.7 Hz, 1H), 8.36 (t, J = 5.3 |
| | 494.3 | Hz, 1H), 8.15 (s, 2H), 7.97-7.88 (m, |
| | | 3H), 7.82 (s, 1H), 7.73 (dd, J = 8.7, |
| | | 1.8 Hz, 1H), 7.52 (dd, J = 8.4, 1.4 Hz, |
| | | 1H), 7.46 (dd, J = 8.4, 1.5 Hz, 1H), |
| | | 7.28 (d, J = 8.7 Hz, 1H), 3.86-3.77 |
| | | (m, 2H), 3.76-3.27 (signal in water, |
| | | 4H), 3.20 (s, 6H), 3.21-3.11 (m, 2H), |
| | | 2.46-2.26 (m, 4H), 2.23 (s, 3H). |
| 3 | 233.7, | 8.51 (d, J = 1.7 Hz, 1H), 8.26 (t, J = |
| | 466.3 | 5.3 Hz, 1H), 8.17 (s, 0.6H), 7.98- |
| | | 7.89 (m, 3H), 7.85 (s, 1H), 7.73 (dd, |
| | | J = 8.7, 1.8 Hz, 1H), 7.56 (dd, J = |
| | | 8.4, 1.5 Hz, 1H), 7.47 (dd, J = 8.4, |
| | | 1.6 Hz, 1H), 7.23 (d, J = 8.7 Hz, 1H), |
| | | 6.69 (br s, 2H), 3.84-3.76 (m, 2H), |
| | | 3.74-3.21 (m, 4H), 3.19-3.11 (m, |
| | | 2H), 2.44-2.25 (signal in water, |
| | | 4H), 2.21 (s, 3H). |
| 4 | 226.2, | 8.91 (s, 2H), 8.20 (d, J = 8.8 Hz, 1H), |
| | 451.3 | 8.00-7.84 (m, 6H), 7.58 (dd, J = 8.4, |
| | | 1.5 Hz, 1H), 7.46 (dd, J = 8.4, 1.5 Hz, |
| | | 1H), 3.88-3.79 (m, 2H), 3.62-3.44 |
| | | (m, 4H), 3.28-3.20 (m, 2H), 2.41- |
| | | 2.32 (m, 4H), 2.24 (s, 3H). |
| 5 | 225.7, | 8.87 (s, 1H), 8.53 (d, J = 5.5 Hz, 1H), |
| | 450.3 | 8.00-7.83 (m, 6H), 7.69 (t, J = 5.2 |
| | | Hz, 1H), 7.58 (dd, J = 8.4, 1.1 Hz, |
| | | 1H), 7.47 (dd, J = 8.4, 1.3 Hz, 1H), |
| | | 6.72 (d, J = 5.6 Hz, 1H), 3.76-3.29 |
| | | (m, 4H), 3.71-3.60 (m, 2H), 3.18 |
| | | (t, J = 7.3 Hz, 2H), 2.45-2.25 |
| | | (m, 4H), 2.21 (s, 3H). |
| 5a | 225.7, | 9.85 (s, 1H), 9.28 (m, 1H), 8.59 (d, J = |
| | 450.2 | 7.0 Hz, 1H), 8.26 (d, J = 8.8 Hz, 1H), |
| | | 8.12 (d, J = 8.7 Hz, 1H), 8.03 (s, 1H), |
| | | 8.00-7.88 (m, 3H), 7.62 (d, J = 8.3 |
| | | Hz, 1H), 7.55 (d, J = 8.4 Hz, 1H), 7.06 |
| | | (d, J = 7.1 Hz, 1H), 3.99-3.88 (m, |
| | | 2H), 3.30-3.05 (m, 8H), 2.78 (s, 3H). |
| 6 | 226.3, | — |
| | 451.4 | |
| 7 | 239.7, | 8.87 (s, 1H), 8.52 (d, J = 5.5 Hz, 1H), |
| | 478.3 | 8.01-7.83 (m, 6H), 7.69 (t, J = 5.1 |
| | | Hz, 1H), 7.58 (d, J = 8.4 Hz, 1H), |
| | | 7.47 (d, J = 9.6 Hz, 1H), 6.72 (d, J = |
| | | 5.5 Hz, 1H), 3.68-3.20 (m, 6H), |
| | | 3.18 (t, J = 7.3 Hz, 2H), 2.69 (p, J = |
| | | 6.5 Hz, 1H), 2.47-2.30 (m, 4H), |
| | | 0.98 (d, J = 6.5 Hz, 6H). |
| 8 | 226.2, | 8.87 (d, J = 5.3 Hz, 1H), 8.55 (d, J = 1.8 |
| | 451.3 | Hz, 1H), 8.10-8.04 (m, 1H), 8.04-7.95 |
| | | (m, 3H), 7.95-7.91 (m, 2H), 7.68 (dd, |
| | | J = 8.4, 1.3 Hz, 1H), 7.48 (dd, J = 8.5, 1.3 |
| | | Hz, 1H), 7.24 (d, J = 5.3 Hz, 1H), 4.62 |
| | | (t, J = 6.5 Hz, 2H), 3.72-3.19 (m, 4H), |
| | | 3.42 (t, J = 6.5 Hz, 2H), 2.44-2.24 (m, |
| | | 4H), 2.20 (s, 3H). |
| 9 | 242.7, | 8.72 (s, 1H), 8.56 (s, 1H), 7.95-7.84 (m, |
| | 484.3 | 4H), 7.77 (d, J = 8.5 Hz, 1H), 7.68 (s, |
| | | 1H), 7.51-7.38 (m, 2H), 7.16 (t, J = |
| | | 6.2 Hz, 1H), 4.09 (q, J = 7.0 Hz, 2H), |
| | | 3.75-3.40 (m, 4H), 3.14 (t, J = 7.1 Hz, |
| | | 2H), 2.45-2.25 (m, 4H), 2.22 (s, 3H). |
| 9a | 242.7, | 11.11 (s, 1H), 9.03 (s, 1H), 8.84 (s, 1H), |
| | 484.3 | 8.71 (s, 1H), 8.16 (d, J = 8.7 Hz, 1H), |
| | | 8.07 (d, J = 8.7 Hz, 1H), 8.01 (s, 1H), |
| | | 7.94 (d, J = 8.5 Hz, 1H), 7.87 (d, J = |
| | | 8.5 Hz, 1H), 7.80 (s, 1H), 7.53 (d, J = |
| | | 8.4 Hz, 2H), 4.34-4.24 (m, 2H), |
| | | 3.80-3.58 (m, 5H), 3.50-3.41 (m, |
| | | 2H), 3.25 (t, J = 7.2 Hz, 2H), 3.16- |
| | | 3.12 (m, 2H), 2.80 (s, 3H) |
| 11 | 226.2, | 9.32 (d, J = 2.0 Hz, 1H), 9.17 (d, J = |
| | 451.3 | 2.1 Hz, 1H), 8.65 (d, J = 5.6 Hz, 1H), |
| | | 8.0-7.86 (m, 5H), 7.58 (d, J = 8.4 |
| | | Hz, 1H), 7.47 (d, J = 9.7 Hz, 1H), |
| | | 6.80 (d, J = 5.7 Hz, 1H), 3.74-3.38 |
| | | (m, 6H), 3.18 (t, J = 7.3 Hz, 2H), |
| | | 2.43-2.26 (m, 4H), 2.21 (s, 3H). |
| 12 | 232.7, | 8.87 (s, 1H), 8.53 (d, J = 5.5 Hz, 1H), |
| | 464.3 | 8.16 (s, 1H), 7.99-7.85 (m, 6H), |
| | | 7.69 (t, J = 5.0 Hz, 1H), 7.58 (dd, |
| | | J = 8.4, 1.5 Hz, 1H), 7.47 (dd, J = |
| | | 8.4, 1.6 Hz, 1H), 6.72 (d, J = 5.6 Hz, |
| | | 1H), 3.72-3.41 (m, 6H), 3.18 (t, J = |
| | | 7.3 Hz, 2H), 2.46-2.32 (m, 6H), |
| | | 1.01 (t, J = 7.2 Hz, 3H). |
| 13 | —, | 8.87 (s, 1H), 8.53 (d, J = 5.5 Hz, 1H), |
| | 437.3 | 8.01-7.85 (m, 6H), 7.68 (t, J = 5.2 |
| | | Hz, 1H), 7.59 (d, J = 8.4 Hz, 1H), |
| | | 7.50 (d, J = 8.4 Hz, 1H), 6.72 (d, J = |
| | | 5.6 Hz, 1H), 3.73-3.36 (m, 10H), |
| | | 3.19 (t, J = 7.2 Hz, 2H). |
| 17 | 259.6, | 8.87 (s, 1H), 8.53 (d, J = 5.5 Hz, 1H), |
| | 518.3 | 8.01-7.85 (m, 6H), 7.70 (s, 1H), |
| | | 7.58 (d, J = 8.5 Hz, 1H), 7.47 (d, J = |
| | | 9.9 Hz, 1H), 6.73 (d, J = 5.6 Hz, 1H), |
| | | 3.79-3.40 (m, 6H), 3.26-3.19 |
| | | (m, 4H), 2.79-2.56 (m, 4H). |
| 18 | 218.6, | 8.87 (s, 1H), 8.53 (d, J = 5.5 Hz, 1H), |
| | 436.2 | 8.00-7.86 (m, 6H), 7.69 (t, J = 5.2 |
| | | Hz, 1H), 7.58 (dd, J = 8.4, 1.2 Hz, |
| | | 1H), 7.47 (dd, J = 8.4, 1.3 Hz, 1H), |
| | | 6.72 (d, J = 5.6 Hz, 1H), 3.73-3.14 |
| | | (signed in water, m, 5H), 3.66 (dd, |
| | | J = 12.6, 7.0 Hz, 2H), 3.19 (t, J = |
| | | 7.3 Hz, 2H), 2.84-2.64 (m, 4H). |
| 19 | 234.1, | 8.87 (d, J = 4.8 Hz, 1H), 8.53 (d, J = |
| | 467.1 | 1.1 Hz, 1H), 8.14 (d, J = 8.8 Hz, 1H), |
| | | 8.07 (dd, J = 8.7, 1.6 Hz, 1H), 7.99- |
| | | 7.87 (m, 4H), 7.70 (d, J = 4.9 Hz, 1H), |
| | | 7.61 (dd, J = 8.3, 1.0 Hz, 1H), 7.47 (dd, |
| | | J = 8.4, 1.1 Hz, 1H), 3.70-3.50 (m, |
| | | 2H), 3.67 (t, J = 7.3 Hz, 2H), 3.26 (t, J = |
| | | 7.3 Hz, 2H), 2.27 (m, 4H), 2.22 (s, 3H). |

### Example 14. Determination of Chemical Stability

Chemical stability of the compounds described herein was determined in human blood plasma.

The determination of stability in human blood plasma was performed using a pooled human blood plasma taken from ten healthy donors. The initial solution of the candidate (10 mM in DMSO) was diluted with the pooled blood plasma to a concentration of 10 μM (test solution). The test solution was incubated in a solid-state thermostat for 4 hours at 37° C. The HPLC method and Agilent1200 chromatograph (Agilent, USA) were used to determine the peak areas of the compounds in the test samples corresponding to the initial test time (before incubation) and the final test time (after incubation in a solid-state thermostat for 4 hours at 37° C.) with preliminary precipitation of proteins using acetonitrile. Chromatographic analysis was performed using a gradient elution mode at a flow rate of 1 ml/min. The amount of substance in the sample expressed in % was determined after the thermostat treatment.

The stability of the compounds was evaluated. The compounds described herein have chemical stability values of more than 75%, i.e., they are chemically stable in the acidic environment of artificial gastric juice and in human blood plasma (Table 7).

TABLE 7
Results of determining chemical stability of the compounds

| Compound number | Stability in blood plasma, % |
|---|---|
| 5 | 100 |
| 9 | 91.1 |
| 9a | 87.9 |
| 18 | 100 |

### Example 15. Determination of Enzymatic Stability

Determining enzymatic stability of the candidates made it possible to evaluate the resistance of the compounds of the present invention to the exposure to biotransformation enzymes.

The enzymatic decomposition rate of the compounds was determined by incubation in a solid-state thermostat at 37° C. of a reaction mixture containing 0.5 mg/ml of pooled S9 fractions of the human liver (XenoTech, USA, cat. # H0610), 10 μm compounds, 2 mM β-nicotinamide adenine dinucleotide (Carbosynth, UK, cat. # NN 10871), and 4 mM magnesium chloride in 0.1 M sodium phosphate buffer (pH=7.4). The reaction was stopped with acetonitrile based on 100 μl of acetonitrile per 100 μl of the reaction mixture. After stopping the reaction, the samples were centrifuged for 10 minutes at 10,000 rpm. The supernatant was analyzed by chromatography using an Agilent1200 chromatograph (Agilent, USA). Chromatographic analysis was performed in a gradient elution mode at a flow rate of 1 ml/min. A logarithm of the peak area of the substance was plotted versus time. The dependent coefficient of this line corresponded to the elimination rate constant (k), which was used to calculate the half-life of the drug (t_{1/2}) and decomposition rate (CLᵢₙₜ):

$$Elimination rate constant ( k ) = ( - gradient )$$

$$Half life ( {t}_{1 \text{/} 2} ) ( min ) = \frac{0.693}{k}$$

$$V ( μL \text{/} mg ) = \frac{volume of incubation ( μL )}{protein in the incubation ( mg )}$$

$$Intrinsic Clearance ( {CL}_{inf} ) ( μL \text{/} min \text{/} mg protein ) = \frac{V \times 0.693}{{t}_{1 \text{/} 2}}$$

The compounds of the present invention showed sufficient resistance to the exposure to biotransformation enzymes and demonstrated the enzymatic decomposition rate (CLᵢₙₜ) of less than 13 l/min/mg. The results are shown in Table 8.

TABLE 8
Results of determining enzymatic stability of the compounds

| Compound number | Stability in S9, CLᵢₙₜ, μl/min/mg |
|---|---|
| 5 | 7.2 |
| 18 | 4.40 |

### Example 16. Determination of Permeability Through a Monolayer of Caco-2 Cells

Determining the permeability through a monolayer of Caco-2 cells makes it possible to assess the ability of the substances to penetrate biological membranes by means of both active and passive transport.

The intestinal epithelial cells (Caco-2) were cultured using filter inserts (0.4 m pores, BD Falcon with High Density) for 21 days, after which the monolayer integrity was checked using a Lucifer Yellow dye (Sigma-Aldrich, USA) according to the standard protocol. When setting up an A→B transfer (“intestinal lumen”—“blood flow” transfer), solutions of the tested substances were introduced in pH 6.5 buffer (HBSS, 10 mM HEPES, 15 mM glucose solution) at a concentration of 10 μM into the upper chamber, while the lower chamber was filled with pH 7.4 buffer (HBSS, 10 mM HEPES, 15 mM Glucose solution, 1% BSA). When setting up a B→A transfer (“blood flow”—“intestinal lumen” transfer), the upper chamber was filled with pH 6.5 buffer, and the solutions of the tested substances were introduced in pH 7.4 buffer at a concentration of 10 μM into the lower chamber. Propranolol, which is a high permeability substance, was used as a control.

After incubation for 2 h at 37° C. in an atmosphere containing 5% CO₂, the amounts of the tested substances in the upper and lower chambers were determined by the HPLC method using an Agilent1200 chromatograph (Agilent, USA) with preliminary precipitation of proteins with acetonitrile. Chromatographic analysis was performed using a gradient elution mode at a flow rate of 1 ml/min. Peak areas corresponding to the compounds were determined on chromatograms. Based on the peak area values of the compound in calibration standards, compound concentrations in the initial solution and in the samples taken from the wells of the upper and lower chambers were determined.

Permeability through a layer of cells (Pₐₚₚ) was calculated based on the formula:

Pₐₚₚ=(C₍ₜ₎*V)/(C_{(θ)}*t*Area), where

Pₐₚₚ is the effective permeability constant, m/s
V is the solution volume (0.8 ml in the A→B test, and 0.2 ml in the B→A test), ml
Area is the surface area of the membrane (0.33 cm²), cm²
t is the incubation time (7,200 s), s
C_{(θ)} is the concentration of the initial solution, μM
C₍ₜ₎ is the concentration of the solution after 2 hours (in the sample from the lower chamber well (test A→B), and in the sample from the upper chamber well (test B→A)), μM.

The efflux coefficient showed the ability of the cells to eliminate the substance from the bloodstream. The value was calculated using the following formula:

efflux=P_{app B-A}/P_{app A-B}, where

P_{app A-B} is the permeability value during direct assay (A→B);
P_{app B-A} is the permeability value during reverse assay (B→A);

The compounds according to the present invention demonstrated a high rate of direct transport “intestinal lumen”—“blood flow,” while the efflux coefficient did not exceed 2, which indicates that the Pgp transporter does not impose any restrictions on the bioavailability of the substance. The results are shown in Table 9.

TABLE 9
Results of determining permeability of the
compounds through a monolayer of Caco-2 cells

| Compound number | A − B, Pₐₚₚ cm/s | Efflux |
|---|---|---|
| 5 | 7.1 | 2.0 |
| 18 | 22.4 | 0.3 |

### Example 17. Antiproliferative Activity with Respect to CDK8-Sensitive Cell Lines In Vitro

The antiproliferative activity of the CDK8 inhibitors according to the present invention was measured in a cell test on a finite MV4-11 cell line (biphenotypic myelomonocytic leukemia, ATCC© CRL-9591™) using an intravital AlamarBlue dye (ThermoFisher, #DAL1100). The cells were grown on an RPMI-1640 medium (PanEco, #C330p) supplemented with 10% FBS (Gibco, #16140-071), then washed and re-seeded on a culture medium with 10% FBS (Gibco, #16140-071) in 96-well culture plates (Corning, #3599) in the amount of ˜10×10³ cells per 100 μl of medium per well. The tested compounds were dissolved in DMSO and diluted with 10% FBS (Gibco, #16140-071) to a final concentration ranging from 0 to 100 μM. Diluted compounds in the amount of 50 μl were then added to each well (final DMSO concentration did not exceed 1%) and incubated at 37° C. in an incubator with 5% CO₂ for 120 h. At the end of the incubation period, 15 μl of AlamarBlue reagent (ThermoFisher, #DAL1100) were added to the wells. The contents of the plates were mixed using an orbital shaker (Biosan, Latvia), and incubated further from 3 to 5 hours at 37° C. in an incubator with 5% C₀₂. The number of living cells was detected using a microplate spectrophotometer (Tecan Infinite M200Pro, Switzerland) by measuring a fluorescence signal at an excitation wavelength (λEx) of 540 nm and emission wavelength (λEm) of 590 nm.

The IC₅₀ value was determined using the Magellan 7.2 program (Tesap, Switzerland) by approximating the experimental points using a four-parameter model with the Levenberg-Marquardt optimization (Table 10).

The CC₅₀ value was determined in a cytotoxicity test. The experiments were performed using HepG2 cells (hepatocellular carcinoma, ATCC© HB-8065™). The cells were seeded into 96-well plates (Corning, #3599) at a concentration of ˜20×10³ cells per 100 μl of medium per well, and incubated for 72 hours with the introduced compounds in the concentration range from 200 to 0.78 μM. Cell viability was evaluated according to the method described above. The results are shown in Table 10.

TABLE 10
Results of specific activity of the compounds in
a cellular antiproliferation test using an MV-4-11
cell line, and results of general toxicity using a
HepG2 cell line. The data are presented using
average activity values obtained during several runs.

| Compound | MV-4-11 | Hep-G2 |
|---|---|---|
| number | IC₅₀, μM | CC₅₀, μM |
| 5 | 0.125 | >60 |
| 7 | 0.091 | 8.9 |
| 8 | 0.602 | 11.2 |
| 9 | 0.106 | 47.3 |
| 11 | 3.297 | 85.1 |
| 12 | 0.079 | 10.6 |
| 13 | 0.057 | ~15 |
| 17 | 0.113 | 17.9 |
| 18 | 0.019 | 11.6 |
| 19 | 2.608 | >200 |

### Example 18. Affinity of the Compounds Towards Recombinant Protein CDK8 in a Complex with Cyclin C In Vitro

The ability of the compounds described herein to bind to the CDK8 protein was determined using the LanthaScreen method (ThermoFisher). A FRET signal was detected, which is proportional to the amount of CDK8-bound fluorescently labeled ligand (Tracer 236), which competes with the inhibitor for an ATP binding site.

The measurements were conducted in a reaction volume (15 μl) using a 384-well plate (Corning, #CLS4513). CDK8/CyclinC enzyme (ThermoFisher, #PR7261B) was mixed with Anti-His-tag-Biotin antibodies (ThermoFisher, #PV6090), Streptavidin-Eu (ThermoFisher, #PV6025), and the resulting mixture was added to the wells of the plate in the amount of 5 μl per well. The final concentrations of the substances were as follows: CDK8/CyclinC—5 nM, Streptavidin-Eu—3 nM, Anti-His-tag-Biotin—3 nM. Staurosporine was used as a control inhibitor, and a 0.1% solution of dimethyl sulfoxide (DMSO) in the reaction buffer containing 250 mM HEPES (pH 7.5), 50 mM MgCl2, 5 mM EGTA, and 0.05% Brij-35 was used as a blank.

Tested inhibitors and controls were added to the corresponding wells in the amount of 5 μl per well. The plate was incubated at room temperature for 20 minutes. At the end of the incubation period, 5 μl of the tracer solution (Alexa Fluor-647 (Kinase Tracer 236, ThermoFisher, #PV5592)) were added to the wells. The final concentration of the tracer was 10 nm. A reaction buffer was used as a negative control instead of the tracer solution. The plate was incubated for 40 minutes at 25° C. Then the TR-FRET signal was measured on a SPARK20 plate reader (Tesap, Switzerland) according to the manufacturer's recommendations, and converted to the amount of kinase-bound tracer. The IC₅₀ value was determined using the SparkControl Magellan 1.2 program (Tesap, Switzerland) by approximating the experimental points using a four-parameter model with the Levenberg-Marquardt optimization (Table 11).

TABLE 11
Results of measuring interactions of the compounds
with CDK8-Cyclin C protein in a biochemical
HTRF test. The data are presented as average
IC₅₀ values obtained during several runs.

| Compound | CDK8-CycC |
|---|---|
| number | IC₅₀, nM |
| 4 | 404.08 |
| 5 | 6.07 |
| 7 | 5.77 |
| 8 | 58.16 |
| 9 | 20.46 |
| 11 | 654.50 |
| 12 | 6.54 |
| 13 | 4.04 |
| 17 | 28.49 |
| 18 | 5.87 |
| 19 | 118.11 |

1. A compound of formula I: 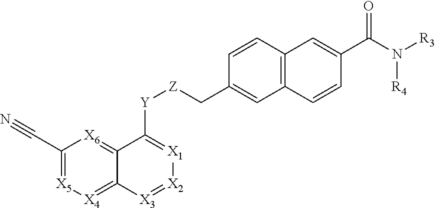
where X₁ represents CR₁, N;
X₂ represents CR₂, N;
X₃, X₄, X₅, X₆ each independently represent CH, N;
R₁ represents H, Hal, C₁-C₆ alkyl, C₃-C₇ cycloalkyl;
R₂ represents H, Hal, C₁-C₆ alkyl, —OR₁₀, —NR₁₁R₁₂,
R₃ and R₄ each independently represent H; C₁-C₆ alkyl unsubstituted or substituted with one or more halogens; C₁-C₆ alkoxy C₁-C₆ alkyl; C₃-C₇ cycloalkyl; C₃-C₇ cycloalkyl C₁-C₃ alkyl; C₆-C₁₀ aryl unsubstituted or substituted with one or more substituents selected from Hal, C₁-C₆ alkyl unsubstituted or substituted with one or more halogens, C₁-C₆, alkoxy; 5-10 membered heteroaryl with 1-4 heteroatoms selected from N, O, and/or S unsubstituted or substituted with one or more substituents selected from Hal, C₁-C₆ alkyl unsubstituted or substituted with one or more halogens, C₁-C₆ alkoxy; 5-6 membered heterocyclyl with 1-2 heteroatoms selected from N, O, and/or S unsubstituted or substituted with one or more substituents selected from Hal; —OH; C₁-C₆ alkoxy; C₁-C₆ alkyl unsubstituted or substituted with one or more halogens, C₁-C₆ alkoxy; or
R₃ and R₄, together with the nitrogen atom to which they are attached, may represent a 4-7 membered heterocyclyl with 1-2 heteroatoms selected from N and/or O, which may be unsubstituted or substituted with one or more substituents selected from the oxo group; —OH; C₁-C₆ alkoxy; C₁-C₆ alkyl unsubstituted or substituted with one or more halogens, C₁-C₆ alkoxy;
Y represents —N(R₁₃)—, —O—, —S—, or —C(O)—;
Z represents —C(R₁₄)₂— or —C(O)—;
R₁₀ represents H, C₁-C₆ alkyl, C₁-C₆ acyl;
R₁₁, R₁₂ each independently represent H, C₁-C₆ alkyl, C₃-C₇ cycloalkyl;
R₁₃ represents H, C₁-C₆ alkyl, C₁-C₆ acyl;
R₁₄ each independently represents H, C₁-C₆ alkyl, C₃-C₇ cycloalkyl; not including compounds where X₁, X₃ represent N; X₂, X₄, X₅, X₆ represent CH; R₃ and R₄ together with the nitrogen atom to which they are attached represent 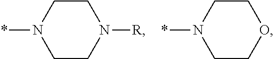
where R represents H, C₁-C₆ alkyl.

2. The compound according to claim 1 wherein if X₁ is CR₁, X₂ is CR₂, and X₃ is N, then X₄, X₅, X₆ represent CH;
if X₁ is CR₁, and X₂, X₃ are N, then X₄, X₅, X₆ represent CH;
if X₂ is CR₂, and X₁, X₃ are N, then X₄, X₅, X₆ represent CH;
if X₁ is CR₁, and X₂, X₄ are N, then X₃, X₅, X₆ represent CH;
if X₁ is CR₁, X₂ is CR₂, and X₃, X₅ are N, then X₄, X₆ represent CH;
if X₁ is CR₁, X₂ is CR₂, and X₃, X₄ are N, then X₅, X₆ represent CH;
if X₁ is CR₁, X₂ is CR₂, and X₃, X₆ are N, then X₄, X₅ represent CH.

3. The compound according to claim 1, which represents a compound of formula II: 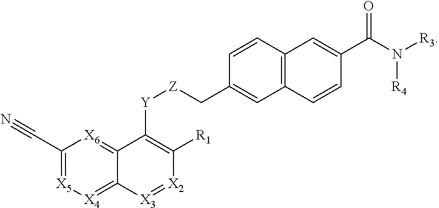
where X₂ represents CR₂, N;
X₃, X₄, X₅, X₆ each independently represent CH, N;
R₁ represents H, Hal, C₁-C₆ alkyl, C₃-C₇ cycloalkyl;
R₂ represents H, Hal, C₁-C₆ alkyl, —OR₁₀, —NR₁₁R₁₂,
R₃ and R₄ each independently represent H; C₁-C₆ alkyl unsubstituted or substituted with one or more halogens; C₁-C₆ alkoxy C₁-C₆ alkyl; C₃-C₇ cycloalkyl; C₃-C₇ cycloalkyl C₁-C₃ alkyl; C₆-C₁₀ aryl unsubstituted or substituted with one or more substituents selected from Hal, C₁-C₆ alkyl unsubstituted or substituted with one or more halogens, C₁-C₆ alkoxy; 5-10 membered heteroaryl with 1-4 heteroatoms selected from N, O, and/or S unsubstituted or substituted with one or more substituents selected from Hal, C₁-C₆ alkyl unsubstituted or substituted with one or more halogens, C₁-C₆ alkoxy; 5-6 membered heterocyclyl with 1-2 heteroatoms selected from N, O, and/or S unsubstituted or substituted with one or more substituents selected from Hal; —OH; C₁-C₆ alkoxy;
C₁-C₆ alkyl unsubstituted or substituted with one or more halogens, C₁-C₆ alkoxy; or
R₃ and R₄ together with the nitrogen atom to which they are attached may represent a 4-7 membered heterocyclyl with 1-2 heteroatoms selected from N and/or O, which may be unsubstituted or substituted with one or more substituents selected from the oxo group; —OH; C₁-C₆ alkoxy; C₁-C₆ alkyl unsubstituted or substituted with one or more halogens, C₁-C₆ alkoxy;
Y represents —N(R₁₃)—, —O—, —S—, or —C(O)—;
Z represents —C(R₁₄)₂— or —C(O)—;
R₁₀ represents H, C₁-C₆ alkyl, C₁-C₆ acyl;
R₁₁, R₁₂ each independently represent H, C₁-C₆ alkyl, C₃-C₇ cycloalkyl;
R₁₃ is H, C₁-C₆ alkyl, C₁-C₆ acyl;
R₁₄ each independently represents H, C₁-C₆ alkyl, C₃-C₇ cycloalkyl.

4. The compound according to claim 1, which represents a compound of formula III: 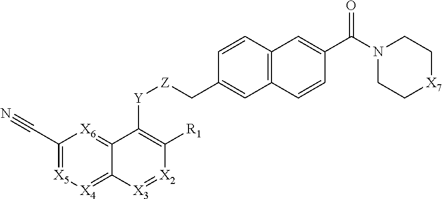
where X₂ represents CR₂, N;
X₃, X₄, X₅, X₆ each independently represent CH, N;
R₁ represents H, Hal, C₁-C₆ alkyl, C₃-C₇ cycloalkyl;
R₂ represents H, Hal, C₁-C₆ alkyl, —OR₁₀, —NR₁₁R₁₂,
X₇ represents —N(R₁₅), —O—;
Y represents —N(R₁₃)—, —O—, —S—, or —C(O)—;
Z represents —C(R₁₄)₂— or —C(O)—;
R₁₀ represents H, C₁-C₆ alkyl, C₁-C₆ acyl;
R₁₁, R₁₂ each independently represent H, C₁-C₆ alkyl, C₃-C₇ cycloalkyl;
R₁₅ represents H, C₁-C₆ alkyl unsubstituted or substituted with one or more halogens, C₁-C₆ alkoxy.

5. The compound according to claim 3, wherein if X₂ is CR₂ and X₃ is N, then X₄, X₅, X₆ represent CH;
if X₂, X₃ are N, then X₄, X₅, X₆ represent CH;
if X₂, X₄ are N, then X₃, X₅, X₆ represent CH;
if X₂ is CR₂, and X₃, X₅ are N, then X₄, X₆ represent CH;
if X₂ is CR₂, and X₃, X₄ are N, then X₅, X₆ represent CH;
if X₂ is CR₂, and X₃, X₆ are N, then X₄, X₅ represent CH.

6. The compound according to claim 1, wherein Y is —NH—, —O—, or —S—; and Z is —CH₂—.

7. The compound according to claim 1, which represents:
2-methoxy-4-((2-(6-(4-methylpiperazin-1-carbonyl)naphth-2-yl)ethyl)amino)quinazolin-6-carbonitrile (1)
2-(dimethylamino)-4-((2-(6-(4-methylpiperazin-1-carbonyl)naphth-2-yl)ethyl)amino)quinazoline-6-carbonitrile (2)
2-amino-4-((2-(6-(4-methylpiperazin-1-carbonyl)naphth-2-yl)ethyl)amino)quinazolin-6-carbonitrile (3)
4-((2-(6-(4-methylpiperazin-1-carbonyl)naphth-2-yl)ethyl)amino)cinnolin-6-carbonitrile (4)
4-((2-(6-(4-methylpiperazin-1-carbonyl)naphth-2-yl)ethyl)amino)quinoline-6-carbonitrile (5)
4-((2-(6-(4-methylpiperazin-1-carbonyl)naphth-2-yl)ethyl)amino)quinoline-6-carbonitrile dihydrochloride (5a)
5-((2-(6-(4-methylpiperazin-1-carbonyl)naphth-2-yl)ethyl)amino)-1,7-naphthyridine-3-carbonitrile (6)
4-((2-(6-isopropylpiperazine-4-carbonyl)naphth-2-yl)ethyl)amino)quinoline-6-carbonitrile (7)
4-(2-(6-(4-methylpiperazin-1-carbonyl)naphth-2-yl)ethoxy)quinoline-6-carbonitrile (8)
3-chloro-4-((2-(6-(4-methylpiperazin-1-carbonyl)naphth-2-yl)ethyl)amino)quinolin-6-carbonitrile (9)
3-chloro-4-((2-(6-(4-methylpiperazin-1-carbonyl)naphth-2-yl)ethyl)amino)quinolin-6-carbonitrile dihydrochloride (9a)
4-((2-(6-(4-methylpiperazin-1-carbonyl)naphth-2-yl)ethyl)amino)-1,7-naphthyridine-6-carbonitrile (10)
5-((2-(6-(4-methylpiperazin-1-carbonyl)naphth-2-yl)ethyl)amino)-1,8-naphthyridine-3-carbo nitrile (11)
4-((2-(6-ethylpiperazin-4-carbonyl)naphth-2-yl)ethyl)amino)quinoline-6-carbonitrile (12)
4-((2-(6-morpholin-4-carbonyl)naphth-2-yl)ethyl)amino)quinoline-6-carbonitrile (13)
4-((3-(6-(4-methylpiperazin-1-carbonyl)naphth-2-yl)propanoyl)quinoline-6-carbonitrile (14)
8-((2-(6-(4-methylpiperazin-1-carbonyl)naphth-2-yl)ethyl)amino)-1,5-naphthyridine-2-carbonitrile (15)
N-(6-cyanoquinolin-4-yl)-2-(6-(4-methylpiperazin-1-carbonyl)naphth-2-yl)acetamide (16)
4-((2-(6-(4-(2,2,2-trifluoroethyl)piperazine-4-carbonyl)naphth-2-yl)ethyl)amino)quinoline-6-carbonitrile (17)
4-((2-(6-(piperazin-1-carbonyl)naphth-2-yl)ethyl)amino)quinoline-6-carbonitrile(18)
4-((2-(6-(4-methylpiperazin-1-carbonyl)naphth-2-yl)ethyl)thio)quinoline-6-carbonitrile (19)

8. A method of inhibiting the biological activity of cyclin-dependent protein kinases CDK8/19 in a subject, achieved by contacting said cyclin-dependent protein kinases CDK8/19 with the compound according to claim 1.

9. A pharmaceutical composition comprising a therapeutically effective amount of the compound according to claim 1, or a pharmaceutically acceptable salt thereof, and one or more pharmaceutically acceptable excipients, and intended for the prevention or treatment of a disease or disorder mediated by the activation of cyclin-dependent protein kinases CDK8/19.

10. The pharmaceutical composition according to claim 9, intended for the prevention or treatment of a disease or disorder mediated by the activation of cyclin-dependent protein kinase CDK8/19, wherein the disease or disorder mediated by the activation of cyclin-dependent protein kinases CDK8/19 is an oncological or hematology-oncology disease.

11. The pharmaceutical composition according to claim 10, wherein the oncological or hematology-oncology disease is selected from the group consisting of colorectal cancer, melanoma, metastatic melanoma, breast cancer, triple negative breast cancer (TNBC), prostate cancer, metastatic ovarian cancer, metastatic gastric cancer, leukemia, acute myeloid leukemia, and pancreatic cancer (PC).

12. A method for treating a disease or disorder mediated by the activation of cyclin-dependent protein kinases CDK8/19, comprising the administration of a therapeutically effective amount of the compound according to claim 1, or a pharmaceutically acceptable salt thereof to a subject in need of such treatment.

13. The method of treating a disease or disorder according to claim 12, wherein the disease or disorder mediated by the activation of cyclin-dependent protein kinases CDK8/19 is an oncological or hematology-oncology disease.

14. The method for treating a disease or disorder according to claim 13, wherein the oncological or hematology-oncology disease is selected from the group consisting of colorectal cancer, melanoma, metastatic melanoma, breast cancer, triple negative breast cancer (TNBC), prostate cancer, metastatic ovarian cancer, metastatic stomach cancer, leukemia, acute myeloid leukemia, and pancreatic cancer (PC).

15. A use of the compound according to claim 1, or a pharmaceutically acceptable salt thereof, for treating a disease or disorder mediated by the activation of cyclin-dependent protein kinases CDK8/19 in a subject in need of such treatment.

16. The use according to claim 15, wherein the disease or disorder mediated by the activation of cyclin-dependent protein kinases CDK8/19 is an oncological or hematology-oncology disease.

17. The use according to claim 16, wherein the oncological or hematology-oncology disease is selected from the group consisting of colorectal cancer, melanoma, metastatic melanoma, breast cancer, triple negative breast cancer (TNBC), prostate cancer, metastatic ovarian cancer, metastatic gastric cancer, leukemia, acute myeloid leukemia, and pancreatic cancer (PC).

18. The compound according to claim 4, wherein if X₂ is CR₂ and X₃ is N, then X₄, X₅, X₆ represent CH;
if X₂, X₃ are N, then X₄, X₅, X₆ represent CH;
if X₂, X₄ are N, then X₃, X₅, X₆ represent CH;
if X₂ is CR₂, and X₃, X₅ are N, then X₄, X₆ represent CH;
if X₂ is CR₂, and X₃, X₄ are N, then X₅, X₆ represent CH;
if X₂ is CR₂, and X₃, X₆ are N, then X₄, X₅ represent CH.

19. The compound according to claim 3, wherein Y is —NH—, —O—, or —S—; and Z is —CH₂—.

20. The compound according to claim 4, wherein Y is —NH—, —O—, or —S—; and Z is —CH₂—.
